(19) United States
(12) Patent Application Publication
(10) Pub. No.: US 2023/0112956 A1
(43) Pub. Date: Apr. 13, 2023

(54) MONOCLONAL ANTIBODY DRY POWDERS

(71) Applicant: BOARD OF REGENTS, THE UNIVERSITY OF TEXAS SYSTEM, Austin, TX (US)

(72) Inventors: Zhengrong CUI, Austin, TX (US); Robert O. WILLIAMS, III, Austin, TX (US); Haiyue XU, Austin, TX (US); Stephanie J. HUFNAGEL, Austin, TX (US); Laura CHOW, Austin, TX (US)

(73) Assignee: BOARD OF REGENTS, THE UNIVERSITY OF TEXAS SYSTEM, Austin, TX (US)

(21) Appl. No.: 17/823,336

(22) Filed: Aug. 30, 2022

Related U.S. Application Data
(60) Provisional application No. 63/238,595, filed on Aug. 30, 2021.

(51) Int. Cl. A61K 9/00; A61K 39/395; A61K 9/16; C07K 16/28; C07K 16/24

(52) U.S. Cl. CPC A61K 9/0075; A61K 39/39591; A61K 9/1623; A61K 9/1617; A61K 9/1694; C07K 16/2818; C07K 16/241

(57) ABSTRACT

Dry powder compositions comprising biologically active antibodies or antibody fragments thereof are provided herein. In some aspects, the present disclosure includes methods of manufacturing these compositions and methods of using them in pulmonary administration or applying them to a tissue surface through a medical dry powder blower/sprayer/insufflator.

This application claims the benefit of priority to U.S. Provisional Application No. 63/238,595, filed on Aug. 30, 2021, the entire contents of which are hereby incorporated by reference.

### BACKGROUND

### 1. Field

The present disclosure relates generally to the field of pharmaceutical formulation, biologics and the manufacture of the same. More particularly, it concerns dry powder compositions that include monoclonal antibodies (mAbs) as well as methods for their use and methods of preparing said powder compositions, such as by thin film freezing.

### 2. Description of Related Art

Today, most protein biologic formulations are stored as liquids or frozen liquids partly due to the relative ease of their manufacture. Liquid storage is not optimal, however, due to poor stability, including aggregation, denaturation, deamidation, oxidation, hydrolysis, and more (Bhambhani and Blue, 2010). This is particularly problematic for large proteins, such as monoclonal antibodies (mAbs), which are expensive to produce and their structure and stability are complex. Antibodies are often provided as intravenous (IV) solutions or high concentration liquids in prefilled syringes that must be transported and stored at cooled temperatures due to their limited stability. Exposure to high or low temperatures without the proper stabilizer(s) can exacerbate protein instability and degradation. Even if stored at the recommended temperatures, mAbs can still experience accidental freezing/thawing (e.g. in an unevenly cooled refrigerator), leading to denaturation and aggregation (Li et al., 2019). Formulating mAbs and other proteins as dry powders can help address this issue of instability and reliance on the cold chain. Dry powders can be reconstituted or directly administered (e.g., using a dry powder inhaler (DPI)) via multiple routes with potential advantageous pharmacologic clinical benefits.

Formulating mAbs and other proteins as dry powders can help to address this issue. Powders can be reconstituted prior to administration for multiple routes. For example, KEYTRUDA (pembrolizumab; anti-PD-1) was originally approved as a powder for reconstitution and IV administration (Merck, 2014). In addition to reconstitution for IV administration, powders can be directly delivered to the lung using a dry powder inhaler (DPI) or the nasal cavity using a device capable of delivery dry powder to the nasal cavity like a nasal dry powder sprayer. Many antibodies with respiratory indications are on the market, with lung cancer being the most common indication (Liang et al., 2020). Omalizumab (anti-IgE) has been studied in the clinic for the treatment of asthma via pulmonary delivery but was formulated as a liquid and is currently only approved for subcutaneous (subQ) injection. Typically delivered via injection, E25 (anti-IgE mAb) was aerosolized for the treatment of allergen-induced bronchospasm in asthmatic patients (Fahy et al., 1999). In contrast to IV E25, aerosolized E25 was well-tolerated but was ineffective in controlling symptoms. When delivered via inhalation, E25 was detected in the broncoalveolar lavage (BAL) and serum of patients. However, the E25 serum concentration was less than 1% of that achieved from IV administration (Fahy et al., 1999; Boulat et al., 1997). Therefore, the lack of efficacy was likely due to E25's low systemic neutralization of IgE, because even if IgE in the lung is neutralized, the pool of IgE from the blood may redistribute to the lungs (Fahy et al., 1999). While systemic absorption was necessary for E25's efficacy in the treatment of asthma, the low systemic absorption of the antibody could be utilized for local therapy in the lungs.

Cetuximab and infliximab have undergone preclinical evaluation for pulmonary delivery.

Cetuximab was also formulated as a liquid, while infliximab was formulated as a powder via spray drying (SD) (Faghihi et al., 2019). SD, as well as the cryogenic techniques such as conventional shelf freeze drying (shelf FD), spray-freeze drying (SFD), and spray-freeze into liquid (SFL) are techniques employed to formulate powders. Unfortunately, the powders produced by those techniques generally suffer from poor aerosol performance and/or reduced activity (Engstrom et al., 2008).

As such, there exists a need for methods for producing powder formulations of antibodies having improved aerosol performance and activity.

### SUMMARY

In some embodiments, the present disclosure provides pharmaceutical compositions and methods of using and preparing compositions comprising antibodies or fragments thereof. These compositions may be prepared using thin film freezing methods to prepare pharmaceutical compositions as dry powders that show one or more improved aerosol properties.

In some aspects, the present disclosure provides pharmaceutical compositions comprise a plurality of drug particles, wherein each drug particle comprises:

(A) an antibody or an antibody fragment;

(B) a sugar or sugar alcohol; and

(C) an amino acid;

wherein the pharmaceutical composition is formulated as a dry powder.

In other aspect, the present disclosure provides pharmaceutical compositions comprise a plurality of drug particles, wherein each drug particle comprises:

(A) an antibody or an antibody fragment;

(B) a sugar or sugar alcohol; and

(C) a mucoadhesive polymer;

wherein the pharmaceutical composition is formulated as a dry powder.

In still another aspect, the present disclosure provides pharmaceutical compositions comprise a plurality of drug particles, wherein each drug particle comprises:

(A) an antibody or an antibody fragment;

(B) a sugar or sugar alcohol; and

(C) a polymer;

wherein the pharmaceutical composition is formulated as a dry powder.

In some embodiments, the dry powder is formulated for administration to the lungs such as by oral inhalation. In other embodiments, the pharmaceutical compositions are formulated for topical administration comprises spraying the pharmaceutical composition onto a surface such as a nasal mucosal surface, an oral mucosal surface, the surface of tumor tissue, a vaginal mucosal surface, skin, an intrapleural space, or a surgical site. In some embodiments, the dry powder is reconstituted into a liquid such as water or saline. In some embodiments, the liquid is formulated for use as an intravenous injection, as a subcutaneous injection, in nebulization, or in spraying into the nasal cavity.

In some embodiments, the pharmaceutical compositions further comprise a buffer. In some embodiments, the buffer is a phosphate buffer or a citrate buffer. In some embodiments, the buffer is a phosphate buffered saline. In some embodiments, the sugar or sugar alcohol is a disaccharide such as lactose, trehalose, or sucrose, or mannitol. In some embodiments, the sugar is lactose. In other embodiments, the sugar is trehalose. In some embodiments, the pharmaceutical compositions further comprise an amino acid. In some embodiments, the amino acid is a canonical amino acid such as a non-polar amino acid. In some embodiments, the amino acid is leucine.

In some embodiments, the pharmaceutical compositions comprise an antibody fragment such as a nanobody or a fragment of antigen-binding (Fab′). In other embodiments, the antibody is a monoclonal antibody. In some embodiments, the antibody is an IgG antibody. In other embodiments, the antibody binds to PD-1 such as an anti-PD-1 antibody. In other embodiments, the antibody binds to CTL4A or PD-L1 such as an anti-CTL4A antibody or an anti-PD-L1 antibody. In other embodiments, the antibody binds to TNF-α such as an anti-TNF-α antibody.

In some embodiments, the pharmaceutical compositions comprise a weight ratio of the sugar to the amino acid from about 1:6 to about 9:1. In some embodiments, the weight ratio is from about 1:2 to about 8:1 of the sugar to the amino acid. In some embodiments, the weight ratio is from about 3:2 to about 3:1. In some embodiments, the weight ratio is about 3:2. In other embodiments, the weight ratio is about 3:1. In other embodiments, the pharmaceutical composition does not comprise an amino acid.

In some embodiments, the pharmaceutical compositions comprise a weight ratio from about 0.1% to about 80% of the antibody relative to the sugar. In some embodiments, the weight ratio is from about 0.25% to about 2.5% of the antibody. In some embodiments, the weight ratio is from about 0.33% to about 1.5% of the antibody. In some embodiments, the weight ratio is about 0.5% of the antibody. In other embodiments, the weight ratio is about 1.0% of the antibody.

In some embodiments, the pharmaceutical compositions comprise an excipient. In some embodiments, the excipient is a pharmaceutically acceptable polymer. In some embodiments, the excipient is chitosan, alginate, gellan, starch, polyacrylate, polyvinylpyrrolidine, or cellulose. In some embodiments, the excipient is polyvinylpyrrolidone such as a polyvinylpyrrolidone with a molecular weight from about 10,000 daltons to about 80,000 daltons. In some embodiments, the molecular weight is about 40,000 daltons.

In some embodiments, the compositions contain particles that have a mass median aerodynamic diameter (MMAD) from about 0.5 μm to about 25.0 μm. In some embodiments, the MMAD is from about 1.0 μm to about 4.0 μm. In some embodiments, the MMAD is from about 1.25 μm to about 3.5 μm. In some embodiments, the MMAD is from about 1.5 μm to about 3.25 μm. In some embodiments, the MMAD is from about 1.5 μm to about 2.5 μm.

In some embodiments, the particles have a geometric standard deviation (GSD) from about 1.0 to about 5.0. In some embodiments, the GSD is from about 1.25 to about 4.0. In some embodiments, the GSD is from about 1.5 to about 3.5. In some embodiments, the GSD is from about 1.75 to about 3.0.

In some embodiments, the pharmaceutical compositions are formulated into a capsule for use in a dry powder inhaler. In some embodiments, the pharmaceutical compositions are formulated into an inhaler. In some embodiments, the pharmaceutical compositions when formulated in an inhaler have a fine powder fraction as a percentage of the recovered dose of greater than 20%. In some embodiments, the fine powder fraction as a percentage of the recovered dose of greater than 40%. In some embodiments, the fine powder fraction as a percentage of the recovered dose of greater than 45%.

In some embodiments, the pharmaceutical compositions when formulated in an inhaler have a fine powder fraction as a percentage of the recovered dose is from about 35% to about 100%. In some embodiments, the fine powder fraction as a percentage of the recovered dose is from about 40% to about 99%. In some embodiments, the fine powder fraction as a percentage of the recovered dose from a dry powder inhaler is from about 45% to about 98%.

In some embodiments, the pharmaceutical compositions when formulated in an inhaler have a fine powder fraction as a percentage of the delivered dose of greater than 50% using an inhaler. In some embodiments, the fine powder fraction as a percentage of the delivered dose of greater than 55%. In some embodiments, the fine powder fraction as a percentage of the delivered dose of greater than 70%.

In some embodiments, the pharmaceutical compositions when formulated in an inhaler have a fine powder fraction as a percentage of the delivered dose is from about 50% to about 100%. In some embodiments, the fine powder fraction as a percentage of the delivered dose is from about 55% to about 99%. In some embodiments, the fine powder fraction as a percentage of the delivered dose is from about 70% to about 98%.

In some embodiments, the antibody retains at least 10% of its unprocessed activity. In some embodiments, the antibody retains at least 50% of its unprocessed activity. In some embodiments, the antibody retains at least 80% of its unprocessed activity.

In some embodiments, at least 50% of the antibodies in the pharmaceutical compositions are in the monomeric form after storage at a storage temperature for a time period. In some embodiments, the pharmaceutical compositions comprise at least at 75% of the antibodies in monomeric form. In some embodiments, the pharmaceutical compositions comprise at least at 80% of the antibodies in monomeric form. In some embodiments, the storage temperature is room temperature. In some embodiments, the storage temperature is from about −180° C. to about 20° C. In some embodiments, the storage temperature is from about −120° C. to about 10° C. In some embodiments, the storage temperature is from about −80° C. to about 5° C. In some embodiments, the storage temperature is from about 10° C. to about 50° C. In some embodiments, the storage temperature is from about 15° C. to about 45° C. In some embodiments, the storage temperature is from about 20° C. to about 40° C.

In some embodiments, the pharmaceutical compositions have been dissolved in a solution such as saline. In some embodiments, the solution is phosphate buffered saline.

In still yet another aspect, the present disclosure provides methods of preparing a pharmaceutical composition described herein comprising:
(A) dissolving:
   (1) an antibody or antibody fragment;
   (2) a sugar or sugar alcohol; and
   (3) an amino acid;
   in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In still another aspect, the present disclosure provides methods of preparing a pharmaceutical composition described herein comprising:
(A) dissolving:
   (1) an antibody or antibody fragment;
   (2) a sugar or sugar alcohol; and
   (3) mucoadhesive polymer;
   in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In still yet another aspect, the present disclosure provides methods of preparing a pharmaceutical composition described herein comprising:
(A) dissolving:
   (1) an antibody or antibody fragment;
   (2) a sugar or sugar alcohol; and
   (3) a pharmaceutically acceptable polymer;
   in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the solvent is water In some embodiments, the solvent is saline. In some embodiments, the solvent is phosphate buffered saline. In some embodiments, the dissolving further comprises an amino acid. In some embodiments, the amino acid is a canonical amino acid. In some embodiments, the amino acid is a non-polar amino acid such as leucine.

In some embodiments, the antibody or antibody fragment, sugar or sugar alcohol, and amino acid are dissolved at a dissolving temperature. In some embodiments, the dissolving temperature is from about −10° C. to about 40° C. In some embodiments, the dissolving temperature is from about −5° C. to about 25° C. In some embodiments, the dissolving temperature is from about 0° C. to about 10° C.

In some embodiments, the pharmaceutical mixture is admixed until the pharmaceutical mixture is clear. In some embodiments, the pharmaceutical mixture comprises a solid content from about 0.05% w/v to about 5% w/v of the antibody and sugar. In some embodiments, the solid content is from about 0.1% w/v to about 2.5% w/v of the antibody and sugar. In some embodiments, the solid content is from about 1% w/v to about 3% w/v of the antibody and sugar. In some embodiments, the solid content is from about 0.15% w/v to about 1.5% w/v of the antibody and sugar. In some embodiments, the solid content is from about 0.2% w/v to about 0.6% w/v of the antibody and sugar. In some embodiments, the solid content is from about 0.5% w/v to about 1.25% w/v of the antibody and sugar.

In some embodiments, the pharmaceutical mixture is applied at a feed rate from about 0.5 mL/min to about 5 mL/min. In some embodiments, the feed rate is from about 1 mL/min to about 3 mL/min. In some embodiments, the feed rate is about 2 mL/min. In some embodiments, the pharmaceutical mixture is exposed to the surface for, and frozen in, about 50 milliseconds to about 5 seconds. In some embodiments, applying comprises spraying or dripping droplets of the pharmaceutical mixture. In some embodiments, the surface temperature is about −180° C. to about 0° C., the diameters of the droplets are about 2-5 millimeters, and the droplets are dropped from a distance about 2 cm to 10 cm from the surface.

In some embodiments, the methods further comprise contacting the droplets with a surface having a temperature differential of at least about 30° C. between the droplets and the surface. In some embodiments, the freezing rate of the droplets is between about 10 K/second and about 10 K/second.

In some embodiments, the methods further comprise removing the solvent from the frozen pharmaceutical mixture to form a dry pharmaceutical mixture. In some embodiments, removing of the solvent comprises lyophilization. In some embodiments, the pharmaceutical mixture is applied with a nozzle such as a needle. In some embodiments, the methods produce a droplet size from about 0.1 mm to about 10 mm in diameter. In some embodiments, the droplet size is from about 0.25 mm to about 5 mm. In some embodiments, the droplet size is from about 0.5 mm to about 2.5 mm.

In some embodiments, the pharmaceutical mixture is applied from a height from about 2 cm to about 50 cm. In some embodiments, the height is from about 5 cm to about 20 cm such as about 10 cm. In some embodiments, the surface temperature is from about −190° C. to 0° C. In some embodiments, the surface temperature is from about −25° C. to about −125° C. such as about −100° C. In some embodiments, the surface is a rotating surface. In some embodiments, the surface is rotating at a speed from about 5 rpm to about 500 rpm. In some embodiments, the surface is rotating at a speed from about 100 rpm to about 400 rpm such as at a speed of about 150 rpm.

In some embodiments, the frozen pharmaceutical composition is dried by lyophilization. In some embodiments, the frozen pharmaceutical composition is dried at a first reduced pressure. In some embodiments, the first reduced pressure is from about 10 mTorr to 500 mTorr. In some embodiments, the first reduced pressure is from about 50 mTorr to about 250 mTorr such as about 100 mTorr. In some embodiments, the frozen pharmaceutical composition is dried at a first reduced temperature. In some embodiments, the first reduced temperature is from about 0° C. to −100° C. In some embodiments, the first reduced temperature is from about −20° C. to about −60° C. such as about −40° C. In some embodiments, the frozen pharmaceutical composition is dried for a primary drying time period from about 3 hours to about 36 hours. In some embodiments, the primary drying time period is from about 6 hours to about 24 hours such as about 20 hours.

In some embodiments, the frozen pharmaceutical composition is dried a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr. In some embodiments, the secondary drying time is at a reduced pressure is from about 50 mTorr to about 250 mTorr such as about 100 mTorr. In some embodiments, the frozen pharmaceutical composition is dried for a secondary drying time at a second reduced temperature. In some embodiments, the second reduced temperature is from about 0° C. to 30° C. In some embodiments, the second reduced temperature is from about 10° C. to about 30° C. such as about 25° C. In some embodiments, the frozen pharmaceutical composition is dried for a second time for a second time period from about 3 hours to about 36 hours. In some embodiments, the second time period is from about 6 hours to about 24 hours such as about 20 hours. In some embodiments, the temperature is changed from the first reduced temperature to the second reduced temperature over a ramping time period. In some embodiments, the ramping time period is from about 3 hours to about 36 hours. In some embodiments, the ramping time period is from about 6 hours to about 24 hours such as about 20 hours.

In some embodiments, the pharmaceutical compositions have a water content of less than 10% such as less than 7.5%. In some embodiments, the water content is less than 5%.

In another aspect, the present disclosure provides pharmaceutical compositions prepared using the methods described herein.

In yet another aspect, the present disclosure provides pharmaceutical compositions comprising a plurality of drug particles; wherein each drug particle comprises:

(A) an anti-PD1 antibody;

(B) trehalose; and

(C) leucine;

wherein the pharmaceutical composition is formulated for administration to the lungs, comprises a weight ratio of trehalose to leucine of 3:1, comprises 1% by weight of the trehalose and leucine, and has a mass median aerodynamic diameter (MMAD) from about 1.0 μm to about 4.0 μm.

In still yet another aspect, the present disclosure provides pharmaceutical compositions comprising a plurality of drug particles; wherein each drug particle comprises:

(A) an anti-TNF-α antibody;

(B) trehalose; and

(C) leucine;

wherein the pharmaceutical composition is formulated for administration to the lungs, comprises a weight ratio of trehalose to leucine of 3:1, comprises 1% by weight of the trehalose and leucine, and has a mass median aerodynamic diameter (MMAD) from about 1.0 μm to about 4.0 μm.

In yet another aspect, the present disclosure provides pharmaceutical compositions comprising a plurality of drug particles; wherein each drug particle comprises:

(A) an anti-CTL4A antibody;

(B) lactose; and

(C) leucine;

wherein the pharmaceutical composition is formulated for administration to the lungs, comprises a weight ratio of lactose to leucine of 3:2, comprises 1% by weight of the lactose and leucine, and has a mass median aerodynamic diameter (MMAD) from about 1.0 μm to about 4.0 μm.

In still another aspect, the present disclosure provides pharmaceutical compositions comprising a plurality of drug particles; wherein each drug particle comprises:

(A) an IgG antibody;

(B) lactose; and

(C) leucine;

wherein the pharmaceutical composition is formulated for administration to the lungs, comprises a weight ratio of lactose to leucine of 3:2, comprises 1% by weight of the lactose and leucine, and has a mass median aerodynamic diameter (MMAD) from about 1.0 μm to about 4.0 μm.

In still yet another aspect, the present disclosure provides pharmaceutical compositions comprising a plurality of drug particles; wherein each drug particle comprises:

(A) an anti-PD1 antibody; and

(B) sucrose;

wherein the pharmaceutical composition is formulated for administration to the lungs, comprises 5% by weight of the sucrose, and has a mass median aerodynamic diameter (MMAD) from about 1.0 μm to about 4.0 μm.

In yet another aspect, the present disclosure provides methods of treating a disease or disorder in a patient in need thereof comprising administering to the patient a therapeutically effective amount of a pharmaceutical composition described herein.

In still yet another aspect, the present disclosure provides pharmaceutical compositions described herein for use in the treatment of a disease or disorder in a patient in need thereof.

In still another aspect, the present disclosure provides uses of a pharmaceutical composition described herein in the manufacture of a medicament for the treatment of a disease or disorder.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C show aerosol performance properties of mAb powder formulations. IgG2a or anti-PD-1 mAb were formulated with lactose/leucine 60:40 in PBS and processed using TFFD or shelf FD. (A-B) Aerosol performance properties of anti-PD1-1-LL-PBS TFFD powder (TFFD) compared to anti-PD1-1-LL-PBS shelf FD powder (Shelf FD). (C) Aerosol performance properties of anti-PD1-1-LL-PBS TFFD powder compared to IgG2a-1-LL-PBS TFFD powder. In B, data are mean±S.D.

FIGS. 2A-2C show characterization of anti-PD1-1-LL-PBS TFFD powder. (A) Powder morphology examined by SEM. Bars are 1 μm. (B) X-ray powder diffraction (XRPD) spectra. (C) Modulated differential scanning calorimetry (mDSC) data.

FIGS. 3A-3E show stability of anti-PD-1 mAbs in liquid or in anti-PD1-LL-PBS TFFD powder. Samples were stored for 6 or 10 weeks and removed from storage conditions immediately prior to analysis using (A-B) SDS-PAGE or (C) SEC. For SDS-PAGE, samples were unfiltered and for SEC samples were prefiltered with 0.45 μm PES filters. (D) mDSC of anti-PD1-1-LL-PBS TFFD powder containing PVP K40. (E) (Relative) moisture content in anti-PD1-1-LL-PBS TFFD powders after 6 or 10 weeks of storage at 4° C., room temperature (RT), or 40° C. as measured using Karl Fischer titration (* indicates difference from time 0). For C and E, data are mean±SD, * indicates significance, and n.s. indicates no significance.

FIGS. 4A & 4B show antigen (PD-1) binding capacity of anti-PD-1 mAbs in liquid before TFFD and after being subjected to TFFD and reconstituted in the initial liquid volume. (A) ELISA data normalized to protein content and (B) quantification of protein loss based on SDS-PAGE (* indicates difference from respective liquid samples (before TFFD). Data are mean f SD, and n.s. indicates no significance.

FIGS. 5A-5E show assessment of protein loss saturability. (A) SDS-PAGE of anti-PD-1 mAbs reconstituted from TFFD powders prepared with higher contents of anti-PD-1 mAbs (2.6, 6.6, or 13.2%, w/w) using lactose/leucine 60:40 as the excipients as compared to liquid before TFFD samples. (B) Quantification of band intensity on SDS-PAGE from A. (C) Monomer content in anti-PD-1 mAbs reconstituted from TFFD powders prepared with higher contents of anti-PD-1 mAbs using lactose/leucine 60:40 as the excipients. (D) SDS-PAGE of anti-PD-1 mAbs reconstituted from TFFD powders prepared with anti-PD-1 mAbs (1% w/w) in a PBS solution containing lactose/leucine 60:40 as the excipients at different volumes. (E) Quantification of band intensity on SDS-PAGE from D. In B, C, and E, data are mean (f SD for B and C) and * indicates significance.

FIGS. 6A-6F show impact of excipient on mAb recovery after TFFD. (A) Lack of visible aggregates in anti-PD-1 mAb TFFD powder prepared with sucrose alone as the excipient in PBS. (B) SDS-PAGE comparing anti-PD-1 mAbs reconstituted from three powders of different compositions (lactose/leucine, 60:40; trehalose/leucine (TL), 75:25; or sucrose (S) alone). (C) Quantification of protein from SDS-PAGE in B (* indicates significance and n.s. indicates no significance, in comparison to respective liquid before TFFD samples). (D) Percent of monomer in anti-PD-1 mAb TFFD powders prepared with trehalose/leucine (75:25) (TL) or sucrose alone (S) as determined by SEC. (E) ELISA data showing the PD-1 binding activity of the anti-PD-1 mAbs reconstituted from the anti-PD-1 mAb TFFD powder prepared with sucrose alone as the excipient. Data were normalized to protein content. (F) Aerosol properties of anti-PD-1 mAb TFFD powder prepared with sucrose alone as the excipient. In C-F, data are mean±SD (n=3).

FIG. 7 shows aerosol properties of anti-PD1-1-M TFFD powder. Data are mean±S.D. (n=3).

FIGS. 8A-8C show characterization of anti-TNF-α mAb dry powder prepared by TFFD with trehalose/leucine 75:25 in PBS. (A) SDS-PAGE showing anti-TNF-α mAbs before and after being subjected to TFFD. (B) Quantification of protein from SDS-PAGE gel in A. (C) Percent of anti-TNF-α mAb monomer before and after being subjected to TFFD.

FIGS. 9A-9D show aerosolization of anti-PD-1 1% (w/w) TFF powder using the Novatech® Talcair™ powder blower. The composition was (FIGS. 9A & 9B) lactose/leucine 60:40 1% (w/v) and (FIGS. 9C & 9D) sucrose 5% (w/v).

### DETAILED DESCRIPTION

In some embodiments, the present disclosure provides antibodies and antibody fragments as dry powders prepared using thin-film freezing (TFF) and characteristics of the resultant powders are investigated herein. In some embodiments, the present disclosure provides methods of making these dry powders using thin-film freezing. These and more details are provided herein.

### I. PHARMACEUTICAL COMPOSITIONS

The present disclosure provides compositions of biologically active antibodies that are prepared using thin-film freezing (TFF). The TFF process is a cryogenic technology newly adapted to the pharmaceutical industry to engineer dry powders with good aerosol performance for pulmonary delivery. Previously, thin-film freezing (TFF) was successfully applied to prepare dry powders of proteins such as lysozyme and lactose dehydrogenase (LDH) while preserving their enzymatic activity, but the aerosol properties of the powders were unknown.

Lung cancer is the most common disease for antibodies being studied for pulmonary administration. A few classes of mAbs have been approved by the FDA for the treatment of lung cancer: anti-epidermal growth factor receptor (anti-EGFR), anti-vascular endothelial growth factor receptor 2 (anti-VEGFR2), anti-vascular endothelial growth factor A (anti-VEGF-A), and anti-programmed cell death protein 1 (anti-PD-1). Guilleminault et al. explored the delivery of cetuximab, an anti-EGFR mAb, via aerosolization into the lungs of mice with orthotopic lung tumors. Aerosolization of cetuximab in solution led to a 4-fold higher lung tumor distribution of cetuximab as compared to IV injection at 2 h. Furthermore, aerosolized cetuximab was able to decrease the mean tumor volume by 37% as compared with saline (p<0.05; Guilleminault et al., 2014), showing that the mAbs retained their functionality after pulmonary delivery. Similar studies and results were produced by Maillet et al., 2011. Hervd et al. administered G6-31, an anti-VEGF mAb, via aerosolization to mice. The plasma C m of G6-31 from the aerosol delivery was about 100-fold less than that of IV delivery, with an estimated bioavailable fraction of 5.1%. Therefore, G6-31 could be used for the treatment of local tumors in the lungs, but likely not systemic metastases or other malignancies. Anti-PD-1 mAbs are unique in that they stimulate the immune system to mount an anti-tumor specific immune response. Unfortunately, they are associated with immune related adverse events (irAEs) that limit their use, and local delivery may improve their tolerability while maintaining their efficacy.

In addition to lung cancer, there are other indications that can benefit from administration via the pulmonary route. Anti-tumor necrosis factor alpha (anti-TNFα) agents have been tested in the clinic for treating idiopathic pulmonary fibrosis (Raghu et al., 2008) and pulmonary sarcoidosis (Baughman et al., 2006; Rossman et al., 2006; Sweiss et al., 2014; Utz et al., 2003), but outcomes have not been promising due to inconsistent efficacy and relapse (Karampitsakos et al., 2019). No anti-TNFα mAbs have been approved to treat pulmonary conditions to date. An anti-TNFα Fab fragment was delivered intrapleurally, reducing talc-induced pleurodosis in rabbits (Cheng et al., 2000). Delivery of an antibody powder via the intrapleural route is a potential application.

Provided herein are dry powder formulations of biologically active monoclonal antibodies (mAbs) that can be made by an ultra-rapid freezing (URF) process. The resulting dry powder formulations have a number of distinct advantages. For example, thin film freezing is an ultra-rapid freezing process (i.e., 100-1000 K/s) that can preserve particle size distribution via accelerating the nucleation rate and the formation of small ice crystals. Biologically active monoclonal antibodies are dropped onto a cryogenically cooled surface to form frozen thin-films within, for example, 50 ms to 5 s. Exposure may comprise spraying or dripping droplets of said biologically active monoclonal antibodies. The freezing surface temperature may be about −180° C. to about 0° C., the diameters of the droplets are about 2-5 millimeters, and the droplets are dropped from a distance about 2 cm to 10 cm from the freezing surface. The method may comprise contacting the droplets with a freezing surface having a temperature differential of at least about 30° C. between the droplets and the surface. The freezing rate of said droplets may be between 10 K/second and 10³ K/second. The method may further comprise removing the solvent from the thin film to form a dry composition. Such as wherein said removing of the solvent comprises lyophilization/sublimation. Other high-speed freezing methods may also be employed. Technologies with slower freezing rate (e.g., conventional shelf freeze-drying) result in phase separation and large ice crystals and thus damage of proteins (e.g., denaturation and/or aggregation). It was shown that, by the use of URF, the compositions can be stabilized such that the mAbs are protected from excessive degradation and components retain substantial biological activity after formulation.

In some cases, formulations include at least one excipient, such as sugar, to provide yet further stabilization. Furthermore, dry powders of the embodiments can comprise a wide variety of antibody-containing compositions. Moreover, it has been demonstrated that the powders of the embodiments can be used to directly administer therapeutic agents, e.g., to the lungs. Thus, the aspects of the present invention provide new pharmaceutical formulations, formulation methods and administration modalities that demonstrate significant advantages over previously compositions and methods that have been used.

In some cases, compositions of the present disclosure comprise mAbs, such as an IgG antibody or an anti-TNF-α antibody. It has been shown that mAbs processed into powders as detailed herein are able to retain substantial activity. Thus, methods and compositions provided herein can be used to stabilize mAbs, such as for storage and/or transportation. Likewise, mAb-containing powders can be directly administered to patients in need thereof (or reconstituted prior to administration).

A. Monoclonal Antibodies (mAbs) and Antibody Fragments

Methods and compositions of the embodiments concern biologically active antibodies. The term “antibody” refers to an intact immunoglobulin of any isotype or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, polyclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2 as either monomeric or as dimeric form. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itself. Antigens are usually proteins and polysaccharides, less frequently also lipids. As used herein, antigens also include immunogens and haptens.

An “Fc” region comprises two heavy chain fragments comprising the CHI and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen. Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies may bind the same epitope within an antigen if they exhibit competitive binding for the antigen.

In some embodiments, the antibody is a monoclonal antibody. In further embodiments, the antibody is an IgG antibody. In some embodiments, the antibody binds to PD-1. In some embodiments, the antibody is an anti-PD-1 antibody. In some embodiments, the antibody binds to PD-L1. In some embodiments, the antibody is an anti-PD-L1 antibody. In some embodiments, the antibody binds to CTL4A. In some embodiments, an anti-CTL4A antibody. In some embodiments, antibody binds to TNF-α. In some embodiments, the antibody is an anti-TNF-α antibody.

In some aspect, the present disclosure provides pharmaceutical compositions comprising a dry powder comprising a plurality of drug particles; wherein each drug particle comprises:

(A) an antibody or an antibody fragment; and

(B) a sugar or sugar alcohol.

In some embodiments, the pharmaceutical composition further comprises a buffer. In some embodiments, the buffer is a phosphate buffer, such as a phosphate buffered saline. In some embodiments, the sugar is a disaccharide, such as lactose, trehalose, or sucrose. In some embodiments, the pharmaceutical composition further comprises an amino acid. In some embodiments, the amino acid is a canonical amino acid. In some embodiments, the amino acid is a non-polar amino acid, such as leucine. In some embodiments, the pharmaceutical composition comprises an antibody fragment, such as a nanobody or an Fab′. In some embodiments, the antibody is a monoclonal antibody. In some embodiments, the antibody is an IgG antibody. In some embodiments, the antibody binds to PD-1. In some embodiments, the antibody is an anti-PD-1 antibody. In some embodiments, the antibody binds to PD-Li. In some embodiments, the antibody is an anti-PD-L1 antibody. In some embodiments, the antibody binds to CTL4A. In some embodiments, the antibody is an anti-CTL4A antibody. In some embodiments, the antibody binds to TNF-α. In some embodiments, the antibody is an anti-TNF-α antibody.

In some embodiments, the pharmaceutical composition comprises a weight ratio of the sugar to the amino acid from about 1:6 to about 9:1, from about 1:2 to about 8:1 of the sugar to the amino acid, from about 3:2 to about 3:1, or from about 1:6, 1:5, 1:4, 1:3, 1:2, 1:1, 2:1, 3:1, 4:1, 5:1, 6:1, 7:1, 8:1, to about 9:1, or any range derivable therein. In other embodiments, the pharmaceutical composition does not comprise an amino acid. In some embodiments, the pharmaceutical composition comprises a weight ratio from about 0.1% to about 80% of the antibody relative to the total excipients, from about 0.25% to about 2.5% of the antibody, from about 0.33% to about 1.5% of the antibody, or from about 0.1%, 0.15%, 0.2%, 0.25%, 0.5%, 1%, 1.5%, 2%, 2.5%, 5%, 10%, 15%, 20%, 30%, 40%, 50%, 60%, 70%, to about 80%, or any range derivable therein. In some embodiments, the weight ratio of the antibody is relative to the total excipients. In other embodiments, the weight ratio of the antibody is relative to the amount of sugar in the composition.

In some embodiments, the drug particles have a mass median aerodynamic diameter (MMAD) from about 0.5 μm to about 10.0 μm, from about 1.0 μm to about 4.0 μm, from about 1.25 μm to about 3.5 μm, from about 1.5 μm to about 3.25 μm, from about 1.5 μm to about 2.5 μm, or from about 0.5 μm, 0.75 μm, 1.0 μm, 1.25 μm, 1.5 μm, 1.75 μm, 2.0 μm, 2.25 μm, 2.5 μm, 2.75 μm, 3.0 μm, 3.25 μm, 3.5 μm, 4.0 μm, 5.0 μm, 6.0 μm, 7.0 μm, 8.0 μm, 9.0 μm, to about 10 μm, or any range derivable therein. In some embodiments, the drug particles have a geometric standard deviation (GSD) from about 1.0 to about 5.0, from about 1.25 to about 4.0, from about 1.5 to about 3.5, from about 1.75 to about 3.0, or from about 1.0, 1.25, 1.5, 1.75, 2.0, 2.25, 2.5, 2.75, 3.0, 4.0, to about 5.0, or any range derivable therein.

In some embodiments, the pharmaceutical composition is formulated into a capsule for use in a dry powder inhaler. In some embodiments, the pharmaceutical composition is formulated into an inhaler. In other embodiments, the pharmaceutical composition is formulated for use in a powder blower or powder sprayer. In some embodiments, the pharmaceutical composition when formulated in an inhaler has a fine powder fraction as a percentage of the recovered dose of greater than 20%, greater than 40%, greater than 45%, from about 35% to about 100%, from about 40% to about 99%, from about 45% to about 98%, or from about 20%, 25%, 30%, 35%, 40%, 45%, 50%, 60%, 70%, 80%, 90%, 91%, 92%, 93%, 94%, 95%, 96%, 97%, 98%, 99%, to about 100%, or any range derivable therein. In some embodiments, the pharmaceutical composition when formulated in an inhaler has a fine powder fraction as a percentage of the delivered dose of greater than 50% using an inhaler, greater than 55%, greater than 70%, from about 50% to about 100%, from about 55% to about 99%, from about 70% to about 98%, or from about 50%, 55%, 60%, 65%, 70%, 75%, 80%, 85%, 90%, 91%, 92%, 93%, 94%, 95%, 96%, 97%, 98%, 99%, to about 100%, or any range derivable therein. In some embodiments, the antibody retains at least 10% of its unprocessed activity, at least 50% of its unprocessed activity, at least 80% of its unprocessed activity, or from about 10%, 200%, 30%, 400%, 50%, 60%, 70%, 80%, 90%, to about 100%, or any range derivable therein.

In some embodiments, at least 50% of the antibodies in the pharmaceutical composition is in the monomeric form after storage at a temperature for a time period. In some embodiments, the pharmaceutical composition comprises at least at 75% of the antibodies in monomeric form, at least at 80% of the antibodies in monomeric form, or from about 50%, 55%, 60%, 65%, 70%, 75%, 80%, 85%, 90%, 95%, to about 100%, or any range derivable therein. In some embodiments, the temperature is room temperature. In some embodiments, the temperature is from about −180° C. to about 20° C., from about −80° C. to about 10° C., from about −10° C. to about 5° C., from about 10° C. to about 50° C., from about 15° C. to about 45° C., from about 20° C. to about 40° C., or from about −180° C., −160° C., −140° C., −120° C., −100° C., −90° C., −80° C., −70° C., −60° C., −40° C., −30° C., −20° C., −10° C., −5° C., 0° C., 5° C., 10° C., 15° C., 20° C., 25° C., 30° C., 35° C., 40° C., 45° C., to about 50° C., or any range derivable therein. In some embodiments, the pharmaceutical composition has been dissolved in water. In some embodiments, the water is saline. In some embodiments, the water is phosphate buffered saline. In some embodiments, the water is a citrate buffer.

B. Excipients

In some aspects, the present disclosure comprises one or more excipients formulated into pharmaceutical compositions. The pharmaceutical compositions comprise one or more excipients such as a sugar or sugar alcohol or an amino acid. Furthermore, the compositions may further comprise one or more additional excipients such as a pharmaceutically acceptable polymer. In some embodiments, the weight ratio of the sugar to the amino acid is from about 1:6 to about 9:1, from about 1:2 to about 8:1, or from about 3:2 to about 3:1. In some embodiments, the weight ratio of the sugar to the amino acid is from about 1:6, 1:5, 1:4, 1:3, 1:2, 1:1, 2:1, 3:1, 4:1, 5:1, 6:1, 7:1, 8:1, to about 9:1, or any range derivable therein. In other embodiments, the pharmaceutical composition does not comprise an amino acid. The pharmaceutical composition may further comprise an amount of either one excipient or a group of excipients from about 20% to about 99.9%, 40% to about 99.5%, or from about 80% to about 99%. The amount of excipients in the pharmaceutical composition may be from about 20%, 25%, 30%, 35%, 40%, 45%, 50%, 55%, 60%, 65%, 70%, 75%, 80%, 82%, 84%, 85%, 85%, 88%, 90%, 92%, 94%, 95%, 96%, 97%, 98%, 99%, 99.2%, 99.4%, 99.5%, 99.6%, 99.8%, or 99.9%, or any range derivable therein.

i. Sugar or Sugar Alcohol

In some aspects, the present disclosure comprises one or more excipients formulated into pharmaceutical compositions. In some embodiments, the excipients used herein are water soluble excipients. These water-soluble excipients include sugars or sugar alcohols such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucin, trileucine, histidine and others.

ii. Amino Acids

In some aspects, the present disclosure provides pharmaceutical compositions comprise one or more amino acids, peptides, or proteins. The amino acids may be one of the canonical amino acids such as glycine, alanine, isoleucine, leucine, proline, valine, phenylalanine, tryptophan, tyrosine, aspartic acid, glutamic acid, arginine, histidine, lysine, serine, threonine, cysteine, methionine, asparagine, or glutamine. The amino acids may also be a non-natural amino acid or a modified amino acid such as a glycosylated or phosphorylated amino acid. The amino acids used herein may be in the form of a polypeptide of multiple amino acids or may be a polypeptide of the same amino acids. In particular, polypeptides of 2, 3, 4, 5, 6, 8, 10, 15, 20, or 25 amino acid residues may be used. In other aspects, larger molecules like amino acids, peptides and proteins are incorporated to facilitate inhalation delivery, including leucin, trileucine, histidine and others.

iii. Buffers

In some aspects, the present disclosure provides composition that comprise one or more buffers. The buffers that may be used in the pharmaceutical composition include a phosphate buffer, a succininate buffer, a citrate buffer, a histidine buffer, or an acetate buffer. The buffer may be used may in an aqueous solution. The aqueous solution may further comprise one or more salts such as a saline solution. The buffer may also further comprise one or more organic solvents in trace amounts.

iv. Other Excipients

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading. It is also contemplated that the present compositions may comprise one or more mucoadhesive polymers. Some non-limiting examples of mucoadhesive polymers include lectins, fimbrin, sodium alginate, sodium carboxymethylcellulose, guar gum, hydroxyethylcellulose, karya gum, methylcellulose, poly(ethylene glycol) (PEG), retene, polyacrylate, starch, chitosan, gellan, or tragacanth.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of ionizable cellulosic polymers include cellulose acetate phthalate and hydroxypropyl methyl cellulose acetate succinate. Finally, some non-limiting examples of neutral cellulosic polymers include hydroxypropyl cellulose, hydroxypropyl methyl cellulose, hydroxyethyl cellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol)(PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

In some embodiments, the compositions described herein contain a pharmaceutically acceptable polymer selected from povidone, copovidone, polyvinyl pyrrolidone, polyvinyl acetate, and SOLUPLUS® (polyvinyl caprolactampolyvinyl acetate-polyethylene glycol graft co-polymer, commercially available from BASF). In particular, the pharmaceutical acceptable polymer may be a copolymer of polyvinyl pyrrolidone and polyvinyl acetate. In particular, the copolymer may comprise about 5-7 vinyl pyrrolidone units to about 3-5 units of vinyl acetate, in particular 6 units of vinyl pyrrolidone and 4 units of vinyl acetate. The number-average of the molecular weight of the polymer may be from about 15,000 to about 20,000 Dalton. The pharmaceutically acceptable polymer may be Kollidan® VA 64 (copovidone, vinylpyrrolidone-vinyl acetate) having a CAS Number of 25086-89-9.

In some embodiments, the excipient used herein is a pharmaceutically acceptable polymer, such as chitosan, alginate, gellan, starch, polyacrylate, polyvinylpyrrolidine, or cellulose. In some embodiments, the excipient is polyvinylpyrrolidone. In some embodiments, the excipient is polyvinylpyrrolidone with a molecular weight from about 10,000 Daltons to about 80,000 Daltons, or from about 10,000 Daltons, 20,000 Daltons, 30,000 Daltons, 40,000 Daltons, 50,000 Daltons, 60,000 Daltons, 70,000 Daltons, to about 80,000 Daltons, or any range derivable therein.

In some aspects, the amount of the excipient in the pharmaceutical composition is from about 0.5% to about 20% w/w, from about 1% to about 10% w/w, from about 2% to about 8% w/w, or from about 2% to about 5% w/w. The amount of the excipient in the precursor solution comprises from about 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein.

### II. THIN FILM FREEZING METHODS

In certain aspects, the present disclosure provides pharmaceutical compositions which may be prepared using a URF process, such as thin-film freezing process. Such methods are described in U.S. Patent Application No. 2010/0221343 and Watts, et al., 2013, both of which are incorporated herein by reference. In some cases, the methods employ an ultra-rapid freezing rate of up to 10,000 K/sec, e.g., at least 1,000, 2,000, 5,000 or 8,000 K/sec. In some embodiments, these methods involve dissolving the components of the pharmaceutical composition into a solvent to form a pharmaceutical mixture. The solvents may be either water or an organic solvent or a mixture of water and organic solvent. In some embodiments, the solvent is water. In some embodiments, the solvent is saline. In some embodiments, the solvent is phosphate buffered saline. In other embodiments, the solvent is citrate buffer, histidine buffer, or succinate buffer.

In some embodiments, an amino acid is further dissolved in the pharmaceutical mixture. In some embodiments, the amino acid is a canonical amino acid. In some embodiments, the amino acid is a non-polar amino acid, such as leucine. In some embodiments, the antibody or antibody fragment, sugar or sugar alcohol, and amino acid are dissolved at a dissolving temperature. In some embodiments, the dissolving temperature is from about −10° C. to about 40° C., from about −5° C. to about 25° C., from about 0° C. to about 10° C., or from about −10° C., −5° C., 0° C., 5° C., 10° C., 15° C., 20° C., 25° C., 30° C., 35° C., to about 40° C., or any range derivable therein. In some embodiments, the pharmaceutical mixture is admixed until the pharmaceutical mixture is clear.

In some embodiments, the pharmaceutical mixture is an aqueous solution that includes an antibody or antibody fragment and a sugar or sugar alcohol. In some embodiments, the pharmaceutical mixture may contain a solid content from about 0.05% w/v to about 5% w/v, from about 0.1% w/v to about 2.5% w/v, from about 1.0% w/v to about 3.0% w/v, from about 0.15% w/v to about 1.5% w/v, from about 0.2% w/v to about 0.6% w/v, or from about 0.5% w/v to about 1.25% w/v of the antibody and sugar, or from about 0.01 w/v, 0.02 w/v, 0.03 w/v, 0.04 w/v, 0.05 w/v, 0.10 w/v, 0.15 w/v, 0.20 w/v, 0.25 w/v, 0.50 w/v, 0.60 w/v, 0.70 w/v, 0.80 w/v, 0.90 w/v, 1.00 w/v, 1.25 w/v, 1.50 w/v, 2.00 w/v, 2.25 w/v, 2.50 w/v, 2.75 w/v, 3.00 w/v, 3.25 w/v, 3.50 w/v, 3.75 w/v, 4.00 w/v, 4.50 w/v, to about 5.00 w/v, or any range derivable therein.

This precursor solution may be deposited on a surface which is at a temperature that causes the pharmaceutical mixture to freeze. In some embodiments, this temperature may be below the freezing point of the solution at ambient pressure. In other embodiments, a reduced pressure may be applied to the surface causing the solution to freeze at a temperature below the ambient pressure's freezing point. In some embodiments, the surface temperature is below 0° C. The surface may also be rotating or moving on a moving conveyer-type system thus allowing the precursor solution to distribute evenly on the surface. Alternatively, the precursor solution may be applied to surface in such a manner to generate an even surface.

After the precursor solution has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a first reduced pressure and/or a first reduced temperature. Such a first reduced temperature may be from 0° C. to about −100° C., from −20° C. to about −60° C., or from about 0° C., −10° C., −20° C., −30° C., −40° C., −50° C., −60° C., −70° C., −80° C., −90° C., to about −100° C., or any range derivable therein. Additionally, the solvent may be removed at a first reduced pressure of from about 10 mTorr to 500 mTorr, from about 50 mTorr to about 250 mTorr, or from about 10 mTorr, 20 mTorr, 30 mTorr, 40 mTorr, 50 mTorr, 100 mTorr, 150 mTorr, 200 mTorr, 250 mTorr, 300 mTorr, 400 mTorr, to about 500 mTorr, or any range derivable therein. In some embodiments, the frozen pharmaceutical composition is dried for a primary drying time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

In some embodiments, the frozen pharmaceutical composition is dried in a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr, from about 50 mTorr to about 250 mTorr, or from about 10 mTorr to 500 mTorr, from about 50 mTorr to about 250 mTorr, or from about 10 mTorr, 20 mTorr, 30 mTorr, 40 mTorr, 50 mTorr, 100 mTorr, 150 mTorr, 200 mTorr, 250 mTorr, 300 mTorr, 400 mTorr, to about 500 mTorr, or any range derivable therein. In some embodiments, the frozen pharmaceutical composition is dried for a secondary drying time at a second reduced temperature. In some embodiments, the second reduced temperature is from about 0° C. to 30° C., from about 10° C. to about 30° C., or from about 0° C., 5° C., 10° C., 15° C., 20° C., 25° C., to about 30° C., or any range derivable therein. In some embodiments, the frozen pharmaceutical composition is dried for a second time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein. In some embodiments, the temperature is changed from the first reduced temperature to the second reduced temperature over a ramping time period. In some embodiments, the ramping time period is from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

In some embodiments, the pharmaceutical composition has a water content of less than 10%, less than 7.5%, or less than 5%, or any range derivable therein.

Such as composition prepared using these methods may exhibit a brittle nature such that the composition is easily sheared into smaller particles when processed through a device. These compositions have high surface areas as well as exhibit improved flowability of the composition. Such flowability may be measured, for example, by the Carr index or other similar measurements. In particular, the Carr index may be measured by comparing the bulk density of the powder with the tapped density of the powder. Such compounds may exhibit a favorable Carr index and may result in the particles being better sheared to give smaller particles when the composition is processed through a secondary device to further process a powder composition.

In one aspect is provided a method for preparing antibodies and antibody fragments as dry powders including: applying antibodies and antibody fragments in liquid to a freezing surface; allowing the liquid to disperse and freeze on the freezing surface thereby forming a thin film.

In some embodiments, the applying includes spraying or dripping droplets of the antibodies and antibody fragments in liquid. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In some embodiments, the method further includes contacting the droplets with a freezing surface having a temperature below the freezing temperature of the liquid (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 degrees Celsius below the freezing temperature). In embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 180 or 200 degrees Celsius.

In some embodiments, the thin film has a thickness of about 5 mm, about 4 mm, about 3 mm, about 2 mm, about 1 mm, less than 3 mm, less than 2 mm, less than 1 mm, less than 500 micrometers. In embodiments, the antibody thin film has a thickness of less than 400 micrometers. In embodiments, the thin film has a thickness of less than 300 micrometers. In embodiments, the thin film has a thickness of less than 200 micrometers. In embodiments, the thin film has a thickness of less than 100 micrometers. In embodiments, the thin film has a thickness of less than 50 micrometers. In embodiments, the antibody thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the thin film has a thickness of about 500 micrometers. In embodiments, the thin film has a thickness of about 400 micrometers. In embodiments, the thin film has a thickness of about 300 micrometers. In embodiments, the thin film has a thickness of about 200 micrometers. In embodiments, the thin film has a thickness of about 100 micrometers. In embodiments, the thin film has a thickness of about 50 micrometers. In embodiments, the thin film has a thickness of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the thin film has a surface area to volume ratio of between about 5 and 500 cm⁻¹. In embodiments, the thin film has a surface area to volume ratio of between 25 and 400 cm⁻¹. In embodiments, the thin film has a surface area to volume ratio of between 25 and 300 cm⁻¹. In embodiments, the thin film has a surface area to volume ratio of between 25 and 200 cm⁻¹. In embodiments, the thin film has a surface area to volume ratio of between 25 and 100 cm⁻¹. In embodiments, the thin film has a surface area to volume ratio of between 100 and 500 cm⁻¹. In embodiments, the thin film has a surface area to volume ratio of between 200 and 500 cm⁻¹. In embodiments, the thin film has a surface area to volume ratio of between 300 and 500 cm⁻¹. In embodiments, the thin film has a surface area to volume ratio of between 400 and 500 cm⁻¹. In embodiments, the thin film has a surface area to volume ratio of between 100 and 400 cm⁻¹. In embodiments, the thin film has a surface area to volume ratio of between 200 and 300 cm⁻¹. In embodiments, the thin film has a surface area to volume ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹. In embodiments, the thin film has a surface area to volume ratio of between about 25 and about 500 cm⁻¹. In embodiments, the thin film has a surface area to volume ratio of between about 25 and about 400 cm⁻¹. In embodiments, the thin film has a surface area to volume ratio of between about 25 and about 300 cm⁻¹. In embodiments, the thin film has a surface area to volume ratio of between about 25 and about 200 cm⁻¹. In embodiments, the thin film has a surface area to volume ratio of between about 25 and about 100 cm⁻¹. In embodiments, the thin 10 film has a surface area to volume ratio of between about 100 and about 500 cm⁻¹. In embodiments, the thin film has a surface area to volume ratio of between about 200 and about 500 cm⁻¹. In embodiments, the thin film has a surface area to volume ratio of between about 300 and about 500 cm⁻¹. In embodiments, the thin film has a surface area to volume ratio of between about 400 and about 500 cm⁻¹. In embodiments, the thin film has a surface area to volume ratio of between about 100 and about 400 cm⁻¹. In embodiments, the thin film has a surface area to volume ratio of between about 200 and about 300 cm⁻¹.

In embodiments, the freezing rate of the droplets is between about 10 K/second and about 10⁵ K/second. In embodiments, the freezing rate of the droplets is between about 10 K/second and about 10⁴ K/second. In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and 10⁵ K/second. In embodiments, the freezing rate of the droplets is between 10 K/second and 10⁴ K/second. In embodiments, the freezing rate of the droplets is between 10 K/second and 10³ K/second. In embodiments, the freezing rate of the droplets is between 10² K/second and 10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, the freezing rate of the droplets is 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, each of the droplets freezes upon contact with the freezing surface in less than about 50, 75, 100, 125, 150, 175, 200, 250, 500, 1,000, or 2,000 milliseconds. In embodiments, each of the droplets freezes upon contact with the freezing surface in less than 50, 75, 100, 125, 150, 175, 200, 250, 500, 1,000, or 2,000 milliseconds.

In embodiments, the droplets have an average diameter between about 0.1 and about 5 mm, between about 2 and about 25 degrees Celsius. In embodiments, the droplets have an average diameter between about 2 and about 4 mm, between about 20 and about 25 degrees Celsius. In embodiments, the droplets have an average diameter between about 1 and about 4 mm, between about 2 and about 25 degrees Celsius. In embodiments, the droplets have an average diameter between about 2 and about 3 mm, between about 2 and about 25 degrees Celsius. In embodiments, the droplets have an average diameter between about 1 and about 3 mm, between about 2 and about 25 degrees Celsius. In embodiments, the droplets have an average diameter between about 1 and about 2 mm, between about 2 and about 25 degrees Celsius. In embodiments, the droplets have an average diameter between about 3 and about 4 mm, between about 2 and about 25 degrees Celsius. In embodiments, the droplets have an average diameter between 0.1 and 5 mm, between 2 and 25 degrees Celsius. In embodiments, the droplets have an average diameter between 2 and 4 mm, between 2 and 25 degrees Celsius. In embodiments, the droplets have an average diameter between 1 and 4 mm, between 2 and 25 degrees Celsius. In embodiments, the droplets have an average diameter between 2 and 3 mm, between 2 and 25 degrees Celsius. In embodiments, the droplets have an average diameter between 1 and 3 mm, between 2 and 25 degrees Celsius. In embodiments, the droplets have an average diameter between 1 and 2 mm, between 2 and 25 degrees Celsius. In embodiments, the droplets have an average diameter between 3 and 4 mm, 30 between 2° and 25° C.

In embodiments, the method further includes removing the solvent (e.g., water or liquid) from the thin film to form a dry powder.

In embodiments, provided is a method of making a dry powder from an antibody thin film (e.g., including an antibody thin film made using a method as described herein), including removing the solvent (e.g., water or liquid) from the thin film to form a dry powder. In embodiments of the methods described herein, the dry powder is a dry powder as described herein, including in an aspect, embodiment, example, table, figure, or claim. In embodiments, a method of making an antibody thin film or a method of making dry powder is used to make a dry powder as described herein, including in an aspect, embodiment, example, table, figure, or claim.

In embodiments, the removing of the solvent includes lyophilization. In embodiments, the removing of the solvent includes lyophilization at temperatures of −20 degrees Celsius or less. In embodiments, the removing of the solvent includes lyophilization at temperatures of −25 degrees Celsius or less. In embodiments, the solvent includes lyophilization at temperatures of −40 degrees Celsius or less. In embodiments, the removing of the solvent includes lyophilization at temperatures of −50 degrees Celsius or less. In embodiments, the removing of the solvent includes lyophilization at temperatures of about −20 degrees Celsius or less. In embodiments, the removing of the solvent includes lyophilization at temperatures of about −25 degrees Celsius or less. In embodiments, the solvent includes lyophilization at temperatures of about −40 degrees Celsius or less. In embodiments, the removing of the solvent includes lyophilization at temperatures of about −50 degrees Celsius or less. Primary drying can be performed at −20° C. to −50° C., and secondary drying can be performed at 4-25° C.

In embodiments, to form an antibody powder, an aqueous antibody composition is first frozen to form a frozen antibody composition, then the frozen water is removed to form the antibody powder. A fast freezing process is used to form the frozen antibody composition. A fast freezing process, as used herein, is a process that can freeze a thin film of liquid (less than about 500 microns or 2-4 mm) in a time of less than or equal to about 5000 milliseconds. In the TFF process liquid droplets fall from a given height and impact, spread, and freeze on a cooled solid substrate. Typically, the substrate is a metal drum that is cooled to below 250° K, or below 200° K or below 150° K. On impact the droplets that are deformed into thin films freeze in a time of between about 70 ms and 3000 ms. The frozen thin films may be removed from the substrate by a stainless steel blade mounted along the rotating drum surface. The frozen thin films are collected in liquid nitrogen to maintain in the frozen state. Further details regarding thin film freezing processes may be found in the paper to Engstrom et al. “Formation of Stable Submicron Protein Particles by Thin Film Freezing” Pharmaceutical Research, Vol. 25, No. 6, June 2008, 1334-1346, which is incorporated herein by reference.

Described herein are compositions and methods for preparing an antibody thin film or a dry antibody by spraying or dripping droplets of a liquid antibody such that the antibody is exposed to an vapor-liquid interface of less than 500 cm⁻¹ area/volume, such as 25 to 500 cm⁻¹ (e.g., less than 50, 100, 150, 200, 250, 300, 400) and contacting the droplet with a freezing surface having a temperature lower than the freezing temperature of the liquid antibody (e.g., has a temperature differential of at least 30° C. between the droplet and the surface), wherein the surface freezes the droplet into a thin film with a thickness of less than about 5 mm, such as about 2-4 mm, about 1 mm, about 500 micrometers (e.g., 450, 400, 350, 300, 250, 200, 150, 100, or 50 micrometers). In embodiments, the method may further include the step of removing the liquid (e.g., solvent, water) from the frozen material to form a dry antibody (e.g., particles). In embodiments, the droplets freeze upon contact with the surface in less than about 50, 75, 100, 125, 150, 175, 200, 250, 500, 1,000, 2,000, or 3000 milliseconds. In embodiments, the droplets freeze upon contact with the surface in less than 50 or 150 milliseconds. In embodiments, the droplet has a diameter between 2 and 5 mm at room temperature. In embodiments, the droplet forms a thin film on the freezing surface of between 50 micrometers and 5 mm, such as 2-4 mm in thickness. In embodiments, the droplets have a cooling rate of between 50-250 K/s. In embodiments, the particles of the dry antibody, after liquid (e.g., solvent or water) removal, have a surface area of at least 10, 15, 25, 50, 75, 100, 125, 150 or 200 m²/gr (e.g., surface area of 10, 15, 25, 50, 75, 100, 125, 150 or 200 m²/gr). Minimizing gas-liquid interface can improve protein stability by limiting the amount of protein that can adsorb to the interface.

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid antibody. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid antibody (e.g., at least 30° C. less than the temperature of the droplet). In embodiments, the liquid antibody further includes one or more excipients selected from sugars, phospholipids, surfactants, polymeric surfactants, vesicles, polymers, including copolymers and homopolymers and biopolymers, dispersion aids, and serum albumin. In embodiments, aggregation of the antibody is less than 10% of the total antibody protein in the composition (e.g., irreversible aggregation). In embodiments, the temperature differential between the droplet and the surface is at least 30° C. In embodiments, the excipients or stabilizers that can be included in the liquid antibodies that are to be frozen as described herein include: cryoprotectants, lyoprotectants, surfactants, fillers, stabilizers, polymers, protease inhibitors, antioxidants and absorption enhancers. Specific nonlimiting examples of excipients that may be included in the antibodies described herein include: sucrose, trehaolose, Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to +10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

As used herein, the term “nanoparticle” has its customary and ordinary definition and refers to discrete particles which behave as a whole unit rather than as individual molecules within the particle. A nanoparticle may have a size from about 1 to about 10,000 nm with ultrafine nanoparticles having a size from 1 nm to 100 nm, fine particles having a size from 100 nm to 2,500 nm, and coarse particles having a size from 2,500 nm to 10,000 nm. In some embodiments, the nanoaggregates described herein may comprise a composition of multiple nanoparticles and have a size from about 10 nm to about 100 μm.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Using Thin-Film Freezing to Develop a Stable Powder with Desirable Aerosol Performance Properties for Pulmonary Delivery of the Monoclonal Antibody, Anti-PD-1, and Anti-TNF-α

A. Material and Methods

1. Materials

In Vivo Plus rat IgG2a clone 2A3 and In Vivo MAb anti-mouse PD-1 clone RMP1-14 monoclonal antibodies (mAbs) were from BioXCell (Lebanon, N.H.). SUPERBLOCK™ blocking buffer in PBS, Immunlon 4 HBX 96-well plates and 2,4,6-trinitrobenzene sulfonic acid (TNBSA) were from Thermo Fischer (Waltham, Mass.). Laemmli 2× buffer, Mini-PROTEAN® TGX™ Precast Gels 4-20%, and Bio-Safe Coomassie G-250 were from Bio-Rad (Hercules, Calif.). Goat Anti-Rat IgG-HRP was from Abcam (Cambridge, UK) and recombinant mouse PD-1 his-tag protein was from R&D Systems (Minneapolis, Minn.). Glycine, TRIZMA@ base, L-leucine, α-lactose monohydrate, D-(+)-trehalose dihydrate, D-mannitol, sodium phosphate monobasic dihydrate, sodium phosphate dibasic, phosphate buffered saline (PBS) with 0.05% TWEEN® 20, pH 7.4, PBS, 3,3′,5,5′-tetramethylbenzidine (TMB), sodium bicarbonate and 2-mercaptoethanol were from Sigma-Aldrich (St. Louis, Mo.). DRISOLY® Methanol Anhydrous was from Mettler Toledo (Columbus, Ohio) and TB syringes with 21G×1 needles were from BD (Franklin Lakes, N.J.). Aluminum pouches were from IMPAK (Los Angeles, Calif.) and silica desiccant was from W. A. Hammond Drierite (Xenia, Ohio). Glass serum vials and plastic low-binding polypropylene cryogenic vials were from DWK Life Sciences (Millville, N.J.). The Dry Powder INSUFFLATOR™ Model DP-4 was from PENNCENTURY™ (Philadelphia, Pa.). QUALI-V®-I HPMC size 3 capsules were from QUALICAPS® (Whitsett, N.C.). Protein ladder was from New England BioLabs (Ipswich, Mass.).

2. Methods

Shelf freeze-drying and thin-film freezing. Samples were prepared by dissolving all components in water or PBS, combined in Eppendorf tubes at the appropriate ratios, and cooled on ice. It is noted that the mAbs received were already in PBS (10 mM, pH 7.0). Therefore, even when the excipients were dissolved in water and then mixed with the mAbs before subject TFF, the formulations still contained PBS, just at a lower level than when the excipients were also dissolved in PBS (10 mM, pH 7.4) before mixing with the mAbs. For TFF, 21G needles on 1 mL syringes were used to apply the samples dropwise onto the rotating drum (150 RPM) of the thin-film freezing device at −100° C. from about 10 cm above. The frozen films were collected in liquid nitrogen and transferred to 5 mL glass or plastic vials, which were half stoppered with rubber stoppers and stored at −80° C. until lyophilization. A VirTis Advantage bench top tray lyophilizer was used (VirTis, Gardiner, N.Y.). The primary drying occurred for 1200 min at −40° C., followed by ramping to 25° C. for 1200 min. Secondary drying occurred at 25° C. for 1200 min. The pressure was held constant at no more than 100 mTorr. For shelf freeze drying (shelf FD), the shelf and the sample were cooled slowly from RT to 40° C. in the lyophilizer. For lyophilization, primary drying occurred for 1200 min held at −40° C., then ramped to 25° C. for 1200 min. Secondary drying occurred at 25° C. for 1200 min. The pressure was held constant at or below 100 mTorr.

Aerosol performance. Dry powder (2-3 mg) was loaded into an HPMC size 3 capsule (VCapst Plus, Lonza, Morristown, N.J.). The capsule was placed in a Plastiape high resistance RS00 DPI that was then attached to a Next Generation Impactor (NGI, Copley Scientific, Nottingham, UK) device containing pans coated with Tween 20 (1.5% in methanol, w/v), and the methanol was allowed to evaporate prior to initiation of the inhalation. The flow rate was 60 L/min for 4 s per actuation, providing a 4 kPa pressure drop across the device. Each stage was collected with 2 mL water with the exception of the throat, which was collected in 4 mL water. For formulations containing leucine, a TNBSA kit was used to determine the leucine content in each stage. Briefly, samples were diluted with 0.1 M sodium bicarbonate, pH 8.5 and TNBSA 0.01% diluted in methanol and incubated at 37° C. for 50 min. The absorbance was read at 335 nm using a Synergy HT plate reader (BioTek, Winooski, Vt.). For formulations lacking leucine, the sugar content (e.g., sucrose) was measured using an XBridge Amide 3.5 μm, 4.6×150 mm column (Waters, Milford, Mass.) on a 1220 Infinity II HPLC (Agilent, Santa Clara, Calif.). The mobile phase was water/acetonitrile 20:80 to 60:40 at a flow rate of 1.0 mL/min for 6 min. The injection volume was 15 μL and the column temperature was 30° C. A 1290 Infinity II ELSD (Agilent, Santa Clara, Calif.) was used to detect the sugar with evaporation and nebulization temperatures of 60° C. and a gas flow rate of 1.6 L/min. The Copley Inhaler Testing Data Analysis Software (v3.10) (CITDAS, Copley Scientific, Nottingham, UK) was used to calculate the aerosol properties, including mass median aerodynamic diameter (MMAD), geometric relative standard deviation (GSD), fine particle fraction (FPF) of recovered dose and of delivered dose. The FPF of recovered dose was calculated as the total amount of leucine collected with an aerodynamic diameter below 5 μm as a percentage of the total amount of excipient collected, while the FPF of delivered dose was calculated as the total amount of leucine collected with an aerodynamic diameter below 5 μm as a percentage of the total amount of excipient deposited on the adapter, the induction port, stages 1-7 and Micro-Orifice Collector (MOC). In the present study, the excipients (leucine or sugar/sugar alcohol), instead of the mAbs, were measured to evaluate the aerosol performance due to the low protein loading in most of the samples and the high volume necessary to collect samples in each stage, making measuring the protein amount at each stage of the NGI difficult due to the low sensitivity of protein assays. Furthermore, leucine is in excess of the mAbs and can interfere with these assays. The mAbs directly have been also previously measured when studying the aerosol performance of formulations with higher protein content and compared the results to that when the excipients were measured, with comparable results.

Size exclusion chromatography (SEC) and Sodium dodecyl sulfate-polyacrylamide gel electrophoresis (SDS-PAGE). Samples were reconstituted with an equal volume of water as the beginning volume prior to TFFD. A small aliquot of sample was then removed and saved for SDS-PAGE while the remainder of the sample was filtered using a 0.45 μm polyethersulfone (PES) filter for SEC. For SEC, an Agilent 300 Å, 2.7 μm, 4.6×150 mm column was used on the 1260 Infinity system (Agilent, Santa Clara, Calif.). The mobile phase was 150 mM sodium phosphate buffer at pH 7, the flow rate was 0.3 mL/min, the time was 10 min, and the wavelength was 220 nm. Unfiltered samples were prepared in Laemmli buffer at 1× and beta-mercaptoethanol 100/(v/v) and heated for 5 min at 95° C. Samples were then loaded into the Mini-PROTEAN® TGX™ Precast Gels 4-20% using the BioRad Tetra cell. The running buffer consisted of 25 mM Trizma, 190 mM glycine, and 0.1% SDS in mEq water. The gel was run for 1 h at 100 V. Afterwards, the gel was washed in ˜50 mL distilled water for 5 min. The wash was repeated for a total of 4 times and the water was removed. Then ˜50 mL of Bio-Safe Coomassie G-250 Stain was added and the gel was allowed to stain for 48 h while rocking. Occasionally throughout the staining (i.e. about 3 times), the gel was microwaved for 20 s to enhance the staining. After staining, the stain was removed and the gel was washed with distilled water for ˜5 h, with the water being replaced about every hour. Quantification was conducted using ImageJ.

Modulated differential scanning calorimetry (mDSC). The mDSC was conducted using a Model Q20 (TA Instruments, New Castle, Del.) differential scanning calorimeter equipped with a refrigerated cooling system (RCS40, TA Instruments, New Castle, Del.). Powder (3-5 mg) was accurately weighed and loaded into Tzero aluminum hermetic crucibles. and A puncture was made in the lid right before the DSC measurement. Samples were first cooled down to −40° C. at a ramp rate of 10° C./min and then ramped up from −40 to 300° C. at a rate of 5° C./min. The rate of dry nitrogen gas flow was 50 mL/min. The scans were performed with a modulation period of 60 s and a modulated amplitude of 1° C. A TA Instruments Trios v.5.1.1.46572 software was used to analyze the data.

X-ray powder diffraction (XRPD). A Rigaku Miniflex 600 II (Rigaku, Woodlands, Tex.) equipped with primary monochromated radiation (Cu K radiation source, X=1.54056 Å) was used to conduct XRPD. Samples were loaded on the sample holder and analyzed in continuous mode under the operating conditions of accelerating voltage of 40 kV at 15 mA, step size of 0.02° over a 2θ range of 5-40°, scan speed of 1°/min, and dwell time of 2 s.

Scanning electron microscopy (SEM). The particle morphology was examined using SEM (Zeiss Supra 40C SEM, Carl Zeiss, Heidenheim an der Brenz, Germany) in the Institute for Cell and Molecular Biology Microscopy and Imaging Facility at the University of Texas at Austin. A small amount of bulk powder (i.e. a flake of TFF powder) was deposited on the specimen stub using double-stick carbon tape. A sputter was used to coat all samples with 15 mm of 60/40 Pd/Pt before capturing the images.

Moisture content measurement. A Mettler Toledo V20 volumetric Karl Fischer (KF) titrator (Columbus, Ohio) was used to measure moisture content in each sample with 5-10 mg of powder diluted into CombiMethanol from Aquastar (Darmstadt, Germany), with 3 independent samples per group. The moisture content of the methanol was also measured. The moisture content was calculated using the equation:

KF=[x(b−a)+y(c−b)]/(b−a)

where KF=Karl Fischer reading (% w/w), x=water content in sample (% w/w), y=water content in anhydrous methanol (% w/w), a=the weight of the empty vial (mg), b=the weight of the vial with the powder (mg), c=the weight of the vial with the powder and the methanol (mg).

Stability. After lyophilization but prior to removal from the lyophilizer, the samples were flushed with nitrogen and the stopper re-cap function was used to cap the vials. Samples were sealed and packaged in aluminum pouches with silica desiccant and the pouches were flushed with nitrogen gas upon sealing. They were stored inside a desiccator at the specified temperatures (i.e. 4° C., room temperature, or 40° C.) for 6 or 10 weeks. At these time points, samples were removed from storage conditions immediately prior to analyzing by SEC, SDS-PAGE, and KF.

Binding capacity. An enzyme-linked immunosorbent assay (ELISA) was used to measure the binding capacity of the anti-PD-1 mAb. Recombinant mouse PD-1 protein was coated at a concentration of 1 μg/mL on Immunlon 4 HBX plates and incubated overnight at 4° C. The next day, plates were washed 4 times with PBS+0.05% Tween 20 (wash buffer) and blocked with SuperBlock™ buffer according to the manufacturer's instructions. The plates were washed again 2-3 times with wash buffer and then samples diluted in PBS to a concentration of approximately 100 ng/mL were added and incubated shaking at room temperature for 2 h. A standard curve was utilized to determine the linear range for the sample dilution. The plate was washed 4 times with wash buffer and then IgG-HRP 1:5000 was added, shaking at room temperature for 1 h. The plate was washed 5× with wash buffer and then TMB was added for 5-15 min. The reaction was stopped with 0.16M sulfuric acid and the plate was read at 450 nm using a Synergy HT plate reader.

Micro-Flow Imaging The anti-PD-1 mAb dry powder samples, upon reconstitution were characterized for subvisible aggregates using micro-flow imaging (MFI5100, ProteinSimple, San Jose, Calif.) equipped with a Bot1 autosampler. Anti-PD-1 mAb dry powders were reconstituted to the original volume using water or PBS to reach a final PBS concentration of 10 mM, pH 7.4. If needed, the samples were further diluted with PBS (10 mM, pH7.4) to a final concentration of 0.1 mg anti-PD-1 mAb per mL. Then, 0.9 mL of the anti-PD-1 mAb solution was transferred into a 96×1 mL well plate (ProteinSimple, San Jose, Calif.). Prior to each sample analysis, a flush/wash cycle was performed to provide a clean baseline and prevent cross contamination between each sample. The flush/wash cycle is a sequence of 5 runs of 0.9 mL of water (HPLC grade, Submicron filtered Safe-Cote®, Fisher) at a flow rate of 6 mL/min. Then the first sample analyzed was 1×PBS, which allowed for a baseline operation to ensure a total particle count 5 300 particles. Sample analysis method used a total of 0.9 mL of sample solution with a 0.2 mL purge. The sample volume analyzed was 0.6 mL at a flow rate of 0.17 mL/min. Each sample was stirred for 3 cycles with a dispensing rate of 1 engineering unit (i.e., selected from level 1-6). MFI View System Suite (MVSS) software (Protein Simple) was used to automate particle characterization. Extrinsic particles (e.g., rubber shards, foreign dust particles) were removed from the data using the “find like particles” feature. Intrinsic particles (e.g., air microbubbles, silicon oil droplets) were removed from the data by applying a filter that counts only particles with an aspect ratio <0.8 or aspect ratio ≥0.8 and intensity standard deviation (STD):100 as previously described (Guo el al., 2021). Liquid controls were prepared similarly except without being subjected to TFFD, and the total aggregate counts were in the range of 1240.6-3283.3 counts/mL (mean f SD, 2069.1±631.6).

Statistical analysis. Statistical analysis was completed with one-way ANOVA tests with Tukey's multiple comparisons tests or student's t test. A P value of ≤0.05 was considered significant. Statistical analyses were performed with GraphPad Prism (San Diego, Calif.).

B. Results and Discussion

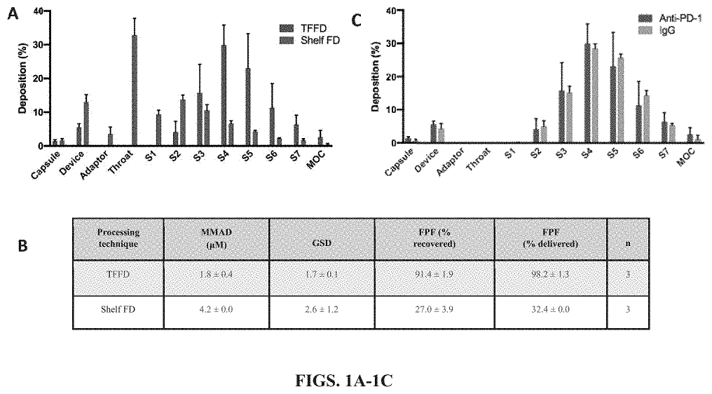

Aerosol performance of TFF mAb powder. Initially, a non-specific mouse IgG was used as a model mAb because all currently FDA-approved mAbs are IgG-based. Initial work identified trehalose/leucine (75:25 w/w) and lactose/leucine (60:40 w/w) to be optimal ratios for formulating proteins with desirable aerosol performance properties. Dry powders of the IgG at a drug loading of 0.5 or 1% (w/w vs. weight of sugar plus leucine) were prepared and evaluated for their aerosol performance. Increasing the mAb content for trehalose/leucine from 0.5% to 1% drastically reduced the FPF recovered (FIGS. 1A-1C). Due to the low antibody content, excipients were measured as a surrogate to determine distribution in the NGI. TFFD powders have been previously shown to have homogeneously mixed active ingredients and excipients (Sahakijpijam et al., 2020b; Thakkar et al., 2018). While all formulations exhibited good aerosol performance, increasing the mAb content for the trehalose/leucine-containing formulations from 0.5% to 1% significantly reduced the FPF recovered (Table 1). When IgG2a dry powders with 1% mAb loading were prepared with lactose/leucine, however, the FPF and all other aerosol properties were retained at desired levels (Table 1).

The use of water versus PBS as the solvent to dissolve excipients for TFF was also explored. Compared to formulations prepared in water, the same formulations prepared in a PBS solution showed improved aerosol properties (indicting that they can be carried deeper into the lungs) and generally had smaller variability between samples (FIGS. 1A-1C). Reproducibility is critical in respiratory delivery since delivery with DPI has high patient-to-patient variability. Because of the aerosol performance and the fact that lactose is currently the only sugar approved by the FDA as a carrier for pulmonary delivery, the TFF 1% lactose/leucine IgG in PBS formulation (IgG-1-LL-PBS) was chosen for further studies. One potential concern was the fact that lactose is a reducing sugar and it may negatively affect the chemical stability of the of mAbs. While using a PBS solution to dissolve excipients had a positive effect on the aerosol performance, it is known that high concentration of salts, especially those in PBS, can negatively affect biologics during freezing and drying (Pikal-Cleland and Carpenter, 2001; Sarciaux et al., 1999; Thorat and Suryanarayanan, 2019). Therefore, it is possible that different mAbs and mAb contents may require avoidance of PBS in the mAb solution before subjecting it to TFFD to better preserve the integrity of the mAbs.

Using the same composition as the IgG-1-LL-PBS, anti-PD-1 mAb dry powders (anti-PD1-1-LL-PBS) were then prepared by TFFD or shelf FD and evaluated their aerosol properties. As shown in FIGS. 1A-B, the TFFD powder demonstrated significantly better aerosol performance as compared to the dry powder with identical composition but prepared by shelf FD, with a smaller MMAD value and larger FPF values (FIG. 1B). Shelf FD is known to produce powders that have relatively low surface area, which likely contributed to poor aerosol performance (Engstrom et al., 2008). This is in large part due to the low freezing rate in shelf-freezing, which allows protein particles to grow over a longer time to reach the frozen state. Conversely, TFFD has a higher freezing rate, which, in addition to the shorter time to reach the frozen state allowing for less particle growth, also leads to more nucleated ice domains with thinner liquid channels. Thinner channels correspond to fewer particle collisions and less particle growth, yielding smaller particles adequate for pulmonary delivery (Hufnagel et al., 2022). SD is another popular technique for producing powders, but exposes the formulation to heat, which can be particularly damaging for biologics, such as mAbs. The powders resulting from SD processing are dense and, thus, often cannot be easily aerosolized (Maa et al., 1999). On the other hand, TFFD powders tend to be porous and have low density, corresponding to readily dispersible powders with high FPFs (Beinborn et al., 2012; Moon et al., 2019; Sahakijpijarn et al., 2020b; Watts et al., 2013). Substituting anti-PD-1 for IgG2a did not significantly affect the aerosol properties of the resultant dry powders (anti-PD1-1-LL-PBS vs. IgG-1-LL-PBS (FIG. 1B vs Table 1, and 1C)), indicating that the TFFD technology can be applied to prepare aerosolizable dry powders of different mAbs.

TABLE 1
Composition and aerosol performance properties of IgG powders prepared by TFFD.

| | Lactose | | | | | | | | |
|---|---|---|---|---|---|---|---|---|---|
| | mAb | | | | Solid | | | | |
| | loading | Ratio | | | content | MMAD | | FPF (% | FPF (% |
| Formulation | (% w/w) | Lactose | Trehalose | Leucine | (% w/v) | (μM) | GSD | recovered) | delivered) |
| IgG-0.5-TL | 0.5 | — | 75 | 25 | 1 | 2.4 ± 0.9 | 3.5 ± 0.6 | 67.3 ± 11.5 | 71.2 ± 12.7 |
| IgG-0.5-TL-PBS | 0.5 | — | 75 | 25 | 1 | 1.7 ± 0.1 | 2.7 ± 0.2 | 78.6 ± 4.9 | 85.5 ± 3.3 |
| IgG-1-TL | 1 | — | 75 | 25 | 1 | 3.2 ± 0.2 | 2.1 ± 0.0 | 47.0 ± 12.1 | 59.8 ± 9.5 |
| IgG-1-TL-PBS | 1 | — | 75 | 25 | 1 | 2.1 ± 0.2 | 2.0 ± 0.3 | 48.3 ± 5.2 | 82.1 ± 11.8 |
| IgG-1-LL | 1 | 60 | — | 40 | 1 | 2.6 ± 0.4 | 1.9 ± 0.1 | 66.7 ± 16.5 | 82.2 ± 12.6 |
| IgG-1-LL-PBS | 1 | 60 | — | 40 | 1 | 1.7 ± 0.1 | 1.9 ± 0.1 | 92.6 ± 1.3 | 97.5 ± 1.3 |

TL = trehalose and leucine; LL = lactose and leucine.
Data are mean ± S.D.
(n = 3).

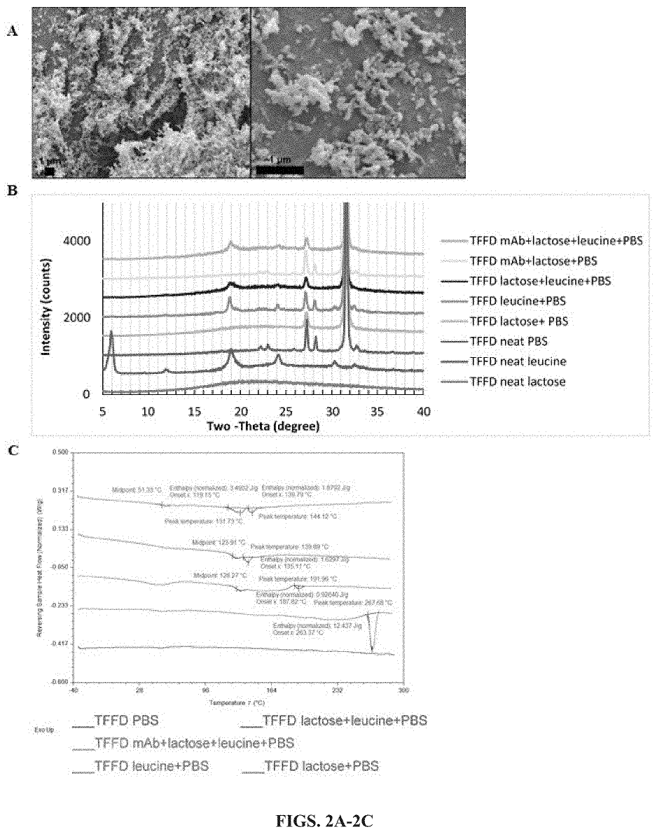

Characterization of the physical properties of anti-PD1-1-LL-PBS powder. After determining that the anti-PD1-1-LL-PBS TFFD powder displayed desired aerosol performance properties, we investigated the physical characteristics of this formulation. SEM showed that the anti-PD1-1-LL-PBS TFFD powder was highly porous and consisted of nanoaggregates (FIG. 2A), which is in alignment with other dry powders produced via TFFD (Engstrom et al., 2008). The moisture content of the anti-PD1-1-LL-PBS powder was 1.5±0.2%. XRPD revealed that lactose was amorphous, while leucine and PBS were crystalline (FIG. 2B). When assessing the physical properties with mDSC, no melting points (MP) for the crystalline PBS could be seen (FIG. 2C). The MPs for sodium chloride and sodium phosphate are >1000° C. and thus were likely out of range (de Jager and Prinsloo, 2001). The mDSC of leucine combined with PBS showed an MP for leucine, confirming leucine's crystalline state. Leucine's MP was around 260° C., decreased from its normal melting point of 300° C. When lactose was combined only with PBS, a T_{g} signal was seen at 128° C., similar to the typical T_{g} of lactose (Huppertz and Gazi, 2016; Roe and Labuza, 2005; Xu et al., 2021). An MP was also present for lactose at 192° C., substantially lower than the typical lactose MP of 220° C. (Wu et al., 2014), which was possibly due to melting point depression by the reduction of the particle size by TFFD, and also showed that lactose in the TFFD powder may be partially crystalline (Rosa et al., 2015). The lactose T_{g} was still present when leucine was added, but the MP was observed at 140° C. Upon addition of anti-PD-1 mAb, the T_{g} was reduced to around 51° C. (a value of 39° C. was observed in one test). Additionally, two MPs were present at 134° C. and 144° C. According to Gombas et al, and Lopez-Pablos et al., the melting peaks in the range of 130-160° C. that were observed in lactose samples are related to the evaporation of water chemically bonded to the lactose molecule (Gombas et al., 2002; López-Pablos et al., 2018).

It is known that crystallization of excipients can lead to phase separation, promote protein aggregation, and limit their stabilizing effects (Chen et al., 2021; Kamerzell et al., 2011; Piedmonte et al., 2007). For example, bovine serum albumin (BSA) spray-dried with leucine showed leucine to be in a largely crystalline state and was phase separated from the protein. Compared to BSA spray-dried with amorphous trehalose, the crystalline leucine formulation had substantially more monomer percent loss after 90 days of storage at 40° C. (Chen et al., 2021). Additionally, BSA spray-dried with leucine exhibited changes in secondary structure and heterogeneity of protein conformation (Chen et al., 2021). While our lactose/leucine formulation contained crystalline PBS and leucine, the lactose was amorphous.

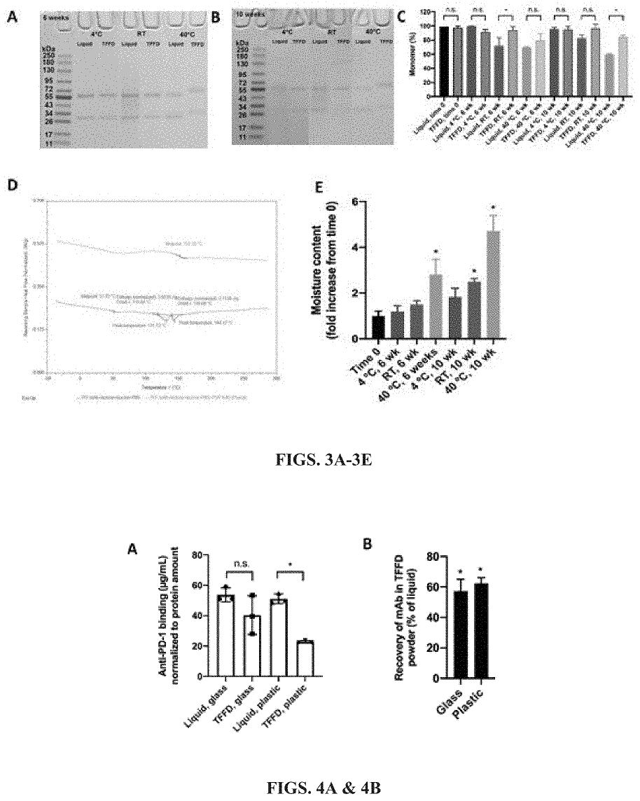

Stability of anti-PD1-1-LL-PBS. To test the stability of the anti-PD-1 mAbs in the anti-PD1-1-LL-PBS TFFD powder, the powder was stored at 4° C., room temperature, or 40° C. for 10 weeks and the integrity of the mAbs was evaluated using SDS-PAGE and SEC. As a control, the stability of anti-PD-1 mAbs in liquid were also tested. As shown in FIGS. 3A-D, the anti-PD-1 mAbs in the TFFD powder were more stable than in the liquid sample at room temperature and 40° C. However, at 40° C., the powder showed a slight upward shift in both bands in the SDS-PAGE image (FIGS. 3A-B), which was also seen as a left shift in SEC peaks (not shown). SEC data revealed that the percent of monomer was lower in the liquid sample stored at room temperature or 40° C. (FIG. 3C) and indicated that the loss was mostly due to degradation. However, the loss of monomer in dry powder stored at 40° C. was more due to aggregation (FIG. 3C), potentially contributing to the increase in protein size in SDS-PAGE and SEC data (FIGS. 3A-B). Both 6- and 10-week stability data showed similar trends in the amount of monomer, with the monomer content decreasing as the storage temperature increased (FIG. 3C). This decrease in monomer was significant in liquid samples at room temperature and 40° C. compared to time 0 (p<0.05 for both temperatures at 6 and 10 weeks). In contrast, loss of monomer in the TFFD powder was seen only when stored at 40° C. and was to a smaller degree than liquid samples (FIG. 3C). Overall, it appears that the anti-PD-1 mAbs in the dry powder was stable after 10 weeks of storage at room temperature, but not at 40° C.

The instability of the anti-PD-1 mAbs in the TFFD powder stored at 40° C. was not unexpected, however, because the T_{g} of the anti-PD1-1-LL-PBS TFFD powder was found to be 39-51° C. (FIG. 2C). To further improve the thermal stability of the anti-PD-1-LL-PBS TFFD powder in the future, a polymer such as PVP K40 that can help increase the T_{g} of the powder may be used. For example, an anti-PD-1 mAb TFFD powder prepared with 5% (w/w) of PVP K40 (i.e., anti-PD1-1-LL-PBS-PVP) showed a T_{g} of 152° C. (FIG. 3D). Symbicort, an FDA-approved product, contains PVP K25, demonstrating the clinical relevance of this class of polymers. To further understand the instability observed when the dry powder was stored at 40° C., the moisture content in the dry powder samples after storage was also measured. Data showed that anti-PD1-1-LL-PBS TFFD powder increased moisture sorption after 6 and 10 weeks of storage at 40° C. (FIG. 3E). Storing the dry powder at room temperature significantly increased the moisture content only after 10 weeks of storage (FIG. 3E). Humidity control and storage conditions are thus important with this hygroscopic powder. Handling of the dry powder in a lower humidity condition and more optimal packaging to minimize moisture uptake during storage are expected to help improve the long-term storage stability of the dry powder without refrigeration or freezing. Nonetheless, data in FIG. 3 suggest the potential of applying the TFFD technology to enable cold chain-free storage of mAbs.

Binding capacity of anti-PD-1 mAb after TFF. The binding capacity of the anti-PD-1 mAbs was measured before and after being subjected to TFFD and reconstitution. The anti-PD1-1-LL-PBS TFFD powder was resuspended in an equal volume of water as the liquid sample (i.e., the same as the liquid prior to TFFD). The binding capacity of the anti-PD-1 mAbs after being subjected to TFFD was not statistically different from that before TFFD, though the variability was high after TFFD (FIG. 4A, glass vial). The high variability may be in part due to interactions of the mAbs with the glass vials. This is supported by the observation that changing the glass to a different material (i.e., plastic) reduced the variability but led to a significant reduction in the binding capacity of the anti-PD-1 mAbs, although the glass vials and plastic vials had similar mAb recovery rates (FIGS. 4A-B). Therefore, the loss of mAb binding capacity using plastic vials is not necessarily inherent to the TFFD process or due to low protein recovery.

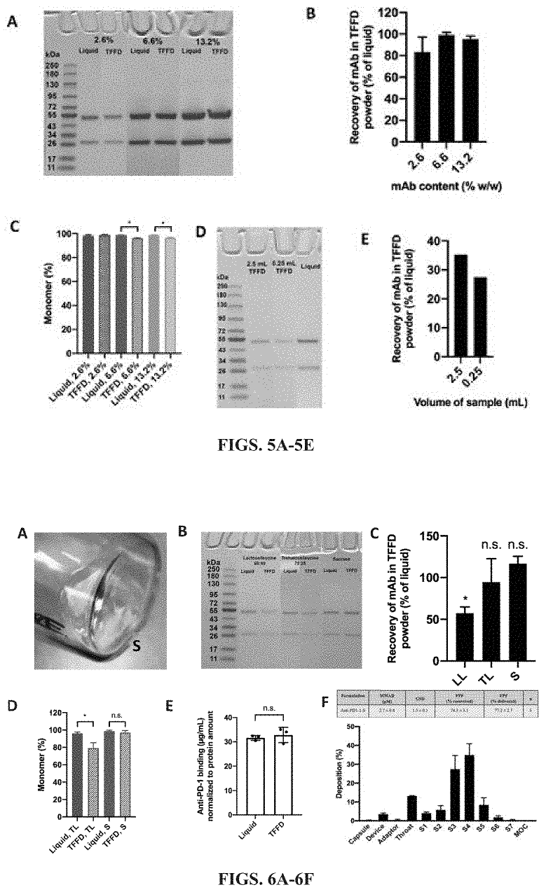

Impact of mAb concentration on protein loss during TFFD. Because complete mAb recovery after TFFD was not achieved (FIG. 4B), we investigated potential reasons underlying the loss of mAbs during TFFD. To test whether the loss could be due to adsorption of mAbs to various surfaces, such as the stainless-steel drum surface of the TFF apparatus, vials, or plastic disposables, we thin-film freeze-dried the anti-PD-1 mAbs at increased mAbs loading (i.e. mAbs to lactose/leucine at 2.6%, 6.6%, and 13.2% (w/w)), while maintaining the content and concentration of other ingredients. As shown in FIGS. 5A-B, increasing the mAb concentration in the solution significantly improved the recovery of the mAbs (to ˜100%), indicating that the mAb loss was likely due to surface adsorption of mAbs during the TFFD process and that the adsorption was saturable. The percentage of monomer in the anti-PD-1 mAb TFFD powder prepared at 6.6% and 13.2% of mAbs remained at 96.1 and 96.4%, respectively, although slightly decreased as compared to its liquid counterpart (FIG. 5C). To test if the protein loss could be saturated by increasing the total volume of solution, we thin-film freeze-dried anti-PD-1 mAbs at the original 1% mAb loading, but using a larger volume (i.e., 0.25 vs. 2.5 mL). As shown in FIGS. 5D-E, increasing the volume of the mAb solution for TFFD only slightly reduced protein loss and was not as effective as increasing the concentration of mAbs (FIG. 5B vs. FIG. 5E). This makes sense, as increasing the volume alone did not saturate the surface area the mAbs were exposed to during TFF because the solution volume and surface area in contact with the solution both increased (i.e., a higher volume of mAb solution corresponds to a higher quantity of liquid in contact with the vial wall, plastics, and the TFF apparatus). This is in contrast to increasing the mAb concentration, in which case the surface area the mAb solution was exposed to stayed the same. This finding further supports that the protein loss observed in the anti-PD1-1-LL-PBS sample was largely due to binding of the mAbs to surfaces during the TFFD process.

Impact of the excipients on protein loss during TFFD. Lactose was largely chosen as the primary excipient when thin-film freeze-drying the anti-PD-1 mAbs in the studies above. It is FDA-approved for respiratory delivery and demonstrated excellent aerosol performance (in combination with leucine). However, lactose is a reducing sugar, a class which is known to contribute to protein instability (Andya et al., 1999; Li et al., 1996) and it was considered the sugar may have contributed to the protein loss. Other excipients such as trehalose and sucrose are also being explored for pulmonary delivery although they are not yet in any FDA-approved products for this route. Leucine was included in the powder formulations because data from previous studies showed that it helps improve the aerosol performance of the TFFD powders (Sahakijpijarn et al., 2020a). Leucine is also in an inhaled product in clinical trials (Waterer et al., 2020). Leucine crystalizes during TFFD, and crystalline forms of molecules are known to be less soluble, which may lead to formulation of visible and subvisible particles during TFFD and reconstitution of the mAbs. In fact, when reconstituting the anti-PD1-1-LL-PBS TFFD powder, some visible particles could be seen. Therefore, i) the effect of using trehalose or sucrose as the primary excipient, with or without leucine to prepare TFFD mAb powders, and ii) their effect on mAb recovery after TFFD were also tested. The visible particles were minimized or eliminated when leucine was not included (FIG. 6A) and thus may be due to leucine's low solubility (˜25 g/L in water at room temperature 25 (Sahakijpijarn et al., 2020a)) or dissolution when in the crystalline state. Keytruda's package insert states to, “discard reconstituted vial if extraneous particulate matter other than translucent to white proteinaceous particles is observed,” implying that some visible protein particles are acceptable even for IV administration (Merck, 2014). The reconstituted formulation has since been discontinued, however. Trehalose/leucine 75:25 (TL) 1% (w/v) and sucrose 5% (w/v) without leucine (S) were also used to formulate anti-PD-1 mAb 1% (w/w) in PBS (anti-PD1-1-TL-PBS and anti-PD1-1-S-PBS) and the protein recovery and percent of monomer were measured. Clearly, excipients impacted the degree of mAb recovery (FIGS. 6B-C). Sucrose as an excipient actually improved the protein recovery of the TFFD samples compared to the liquid samples. Additionally, trehalose/leucine had better recovery of protein than lactose/leucine (FIGS. 6B-C). Furthermore, sucrose as an excipient did not decrease the percent of monomer in the TFFD powder, while trehalose/leucine did (FIG. 6D). Due to the excellent protein and monomer recovery in the sucrose formulation (FIGS. 6B-D), the binding activity and aerosol performance were measured as well. The sucrose formulation maintained similar anti-PD-1 mAb binding before and after processing with TFFD and reconstitution (FIG. 6E). Furthermore, the sucrose formulation had good aerosol performance even without leucine (FIG. 6F). Without wishing to be bound by any theory, it is believed that leucine could be responsible in part to protein loss, with 0% leucine having the best recovery (FIGS. 6B-C), as well as no visible aggregates upon reconstitution (FIG. 6A). Therefore, future formulation efforts should be focused on identifying the proper leucine content in the powder so that the protein loss will be minimized, while the resultant dry powder still has good aerosol performance properties.

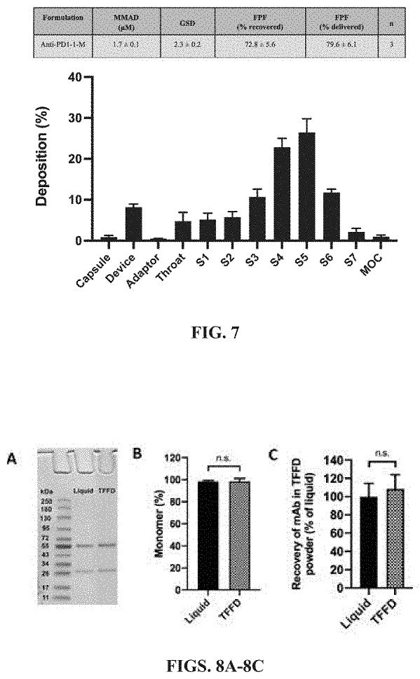

Impact of excipient on subvisible protein aggregate formation during TFFD Aggregation is a major consideration in preparing dry powder formulations of proteins, as it can lead to immunogenicity that can be detrimental to patient health (Ratanji et al., 2014). Additionally, it is known that increasing mAb concentration can often reduce their stability during freeze-drying (Wang et al., 2007). As mentioned above, some visible particles were observed, particularly in the lactose/leucine formulation. All anti-PD1 mAb formulations mentioned above as well as two new anti-PD-1 mAb formulations prepared with mannitol with or without leucine as excipient(s) were also evaluated for subvisible aggregates using MFI. While formulations prepared with PBS (10 mM, pH7.4) as the solvent had superior aerosol performance to those prepared without PBS (TABLE 1), MFI showed that across all formulations, formulations that were prepared with PBS as the solvent had significantly more subvisible aggregates (TABLE 2). Interestingly, increasing the mAb content (1%-13.2%) did not significantly increase the total subvisible aggregate count (TABLE 2). Aggregation is known to occur at the air/liquid interface (Hufnagel et al., 2022). Without wishing to be bound by any theory, it is believed that this interface became saturated, and increasing the protein loading did not increase the amount of protein at the interface. Therefore, the protein loading can be increased to prevent loss of protein (FIG. 5) without increasing subvisible aggregate count (TABLE 2). Lastly, mannitol was the excipient that induced the least amount of subvisible aggregates (TABLE 2), with the least aggregation observed in the TFFD powder formulation prepared with mannitol but without leucine (TABLE 2). Data in FIG. 7 showed that TFFD powder prepared with mannitol as the excipient also showed good aerosol performance properties.

TABLE 2
Subvisible aggregates in anti-PD1 mAb TFFD powders after reconstitution.

| | Size (ECD) range summary (μm) - concentration (#/mL) | | | | | | | |
|---|---|---|---|---|---|---|---|---|
| | | 2 ≤ | 5 ≤ | 10 ≤ | 25 ≤ | 50 ≤ | 75 ≤ | 100 ≤ |
| Formulations | Total | x < 5 | x < 10 | x < 25 | x < 50 | x < 75 | x < 100 | x < 300 |
| Anti-PD1-1-LL-PBS | 794098.8 ± | 477838.4 ± | 218988.1 ± | 93173.2 ± | 3882.2 ± | 184.8 ± | 24.6 ± | 7.6 ± |
| | 112883.1* | 61886.2 | 34850.5 | 26640.5 | 1324.7 | 34.5 | 11.5 | 8.7 |
| Anti-PD1-1-LL | 43134.1 ± | 38798.1 ± | 3133.0 ± | 975.0 ± | 179.5 ± | 39.9 ± | 7.6 ± | 1.0 ± |
| | 12731.7 | 11307.8 | 1298.3 | 853.1 | 249.5 | 56.8 | 13.2 | 1.6 |
| Anti-PD1-1-TL-PBS | 592830.6 ± | 399774.2 ± | 142009.7 ± | 48784.1 ± | 2121.9 ± | 108.3 ± | 20.9 ± | 11.4 ± |
| | 123482.9* | 49931.9 | 50773.9 | 23800.0 | 885.8 | 54.4 | 28.8 | 17.3 |
| Anti-PD1-1-TL | 32377.5 ± | 28593.9 ± | 2758.9 ± | 820.1 ± | 165.8 ± | 24.6 ± | 8.5 ± | 5.7 ± |
| | 11790.1 | 9647.5 | 1336.6 | 588.2 | 245.4 | 40.2 | 12.4 | 9.8 |
| Anti-PD1-1-S-PBS | 466405.0 ± | 340423.3 ± | 102254.6 ± | 23442.8 ± | 250.2 ± | 27.5 ± | 6.6 ± | 0.0 ± |
| | 67632.3* | 24080.4 | 31384.8 | 12306.6 | 93.1 | 15.7 | 9.1 | 0.0 |
| Anti-PD1-1-S | 42672.2 ± | 36822.5 ± | 4776.4 ± | 3631.8 ± | 39.9 ± | 5.7 ± | 0.0 ± | 0.0 ± |
| | 12098.5 | 9691.7 | 2227.4 | 3846.1 | 12.1 | 4.0 | 0.0 | 0.0 |
| Anti-PD1-1-LL-PBS | 794098.8 ± | 477838.4 ± | 218988.1 ± | 93173.2 ± | 3882.2 ± | 184.8 ± | 24.6 ± | 7.6 ± |
| | 112883.1^{a} | 61886.2 | 34850.5 | 26640.5 | 1324.7 | 34.5 | 11.5 | 8.7 |
| Anti-PD1-1-6.6-LL-PBS | 564446.8 ± | 376555.9 ± | 137292.8 ± | 47801.8 ± | 2462.7 ± | 233.1 ± | 57.8 ± | 42.6 ± |
| | 183627.1^{a} | 87193.3 | 65224.6 | 38810.0 | 1608.5 | 181.5 | 48.9 | 39.4 |
| Anti-PD1-1-13.2-LL-PB S | 725153.3 ± | 396621.0 ± | 203929.9 ± | 117366.0 ± | 7103.8 ± | 124.0 ± | 7.1 ± | 1.4 ± |
| | 11901.5^{a} | 9050.2 | 5544.6 | 12522.3 | 2729.6 | 143.2 | 10.1 | 2.0 |
| Anti-PD1-1-ML | 31482.1 ± | 29035.6 ± | 2055.4 ± | 367.4 ± | 19.0 ± | 4.7 ± | 0.0 ± | 0.0 ± |
| | 27345.0^{n.s.} | 27029.0 | 354.6 | 30.4 | 14.4 | 1.6 | 0.0 | 0.0 |
| Anti-PD1-1-M | 8977.2 ± | 8124.4 ± | 696.5 ± | 127.9 ± | 19.0 ± | 2.8 ± | 2.8 ± | 3.8 ± |
| | 2510.7^{n.s.} | 2234.8 | 237.8 | 47.0 | 5.9 | 4.9 | 2.8 | 1.6 |

Shown are MFI counting data comparing subvisible aggregates in anti-PD1 mAb dry powders of different compositions (lactose/leucine (LL), 60:40; trehalose/leucine (TL), 75:25; sucrose (S) alone; mannitol/leucine (ML), 90:10; or mannitol (M) alone, with or without PBS).
For powders prepared with anti-PD1 mAbs at 1% (w/w) with or without PBS, the total particle counts in samples prepared with PBS were significantly higher than in respective samples prepared without PBS (*p < 0.05, t-test).
For powders prepared with different amount of anti-PD1 mAbs (i.e., 1, 6.6, or 13.2%, w/w), one-way ANOVA did not reveal any difference in the total particle counts among them (^{a}p = 0.21).
The n.s. indicates no significance between the total particle counts in anti-PD1-1-ML and anti-PD1-1-M.
Data are mean ± SD (n = 3).
Notes:
Aggregates of >300 μm were not detected, and the MFI did not count particles smaller than 2 μm. Statistical analyses were only done with the total particle counts.

Thin-film freeze-drying of anti-TNF-α mAb. Finally, anti-TNF-α mAb was also formulated at 1% (w/w) with trehalose/leucine 75:25 in PBS (anti-TNFα-1-TL-PBS). Interestingly, there was a full recovery of the protein (FIGS. 8A-B). When anti-PD-1 mAb was formulated at 1% with trehalose/leucine 75:25, the monomer content decreased from 96.0 to 79.2% after TFFD (FIG. 6C). However, when the mAb was changed to anti-TNF-α mAb while other formulation parameters were the same, the monomer percentage did not decrease after TFFD (FIG. 8C). Therefore, as expected, the characteristics of each mAb, including the composition of the liquid in which the mAb is dissolved, can also impact the recovery and stability of the protein during TFFD and each mAb requires its own formulation optimization.

Besides anti-PD1 mAbs and anti-TNF-αc mAbs, TFFD was also successfully utilized to prepare aerosolizable dry powders of other mAbs and antibody fragments (Fabs), including AUG-3387 (Emig et al., 2021), as well as other proprietary clinical grade mAbs and Fabs, with protein to excipient ratios as high as 30-40% (w/w) without significant protein aggregation as confirmed using MFI, SEC, and dynamic light scattering.

### Example 2—Aerosolization of Thin-Film Freeze-Dried Monoclonal Antibody Powder Using a Medical Powder Blower

A. Methods

Samples were prepared with 1% (w/w) In Vivo MAb anti-mouse PD-1 clone RMP1-14 monoclonal antibody (BioXCell, Lebanon, N.H.). Two different compositions were prepared: lactose/leucine 60:40 with 1% (w/v) solids content or sucrose 5% (w/v). Samples were prepared by dissolving all components in PBS, combined in Eppendorf tubes at the appropriate ratios, and cooled on ice. The total liquid sample volume was 2.5 mL. For thin-film freezing (TFF), 21G needles on 1 mL syringes were used to apply the samples dropwise onto the rotating drum (150 rpm) of the TFF device at −100° C. from about 10 cm above. The frozen films were collected in liquid nitrogen and transferred to 5 mL serum glass vials (DWK Life Sciences, Rockwood, Tenn.), which were half stoppered with rubber stoppers and stored at −80° C. until lyophilization. A VirTis Advantage bench top tray lyophilizer was used (VirTis, Gardiner, N.Y.). For lyophilization, primary drying occurred for 1200 min held at −40° C., then ramped to 25° C. for 1200 min. Secondary drying occurred at 25° C. for 1200 min. The pressure was held constant at or below 100 mTorr. The 2.5 mL liquid volume was converted to about 5 mL of powder, filling the vials.

A NOVATECH® TALCAIR™ powder blower (Boston Medical Group, Shrewsbury, Mass.) was used to aerosolize the powder. The powder blower is an FDA-approved device for spraying Talc powder into the intrapleural space. It was chosen to test the feasibility of spraying the thin-film freeze-dried mAb powders using a medical powder blower. The device was held approximately 5 cm from black poster board and 1 actuation of the bulb was executed per photo. The photos of the powder stuck to the poster were taken immediately after aerosolization (i.e. about 10 s).

B. Results

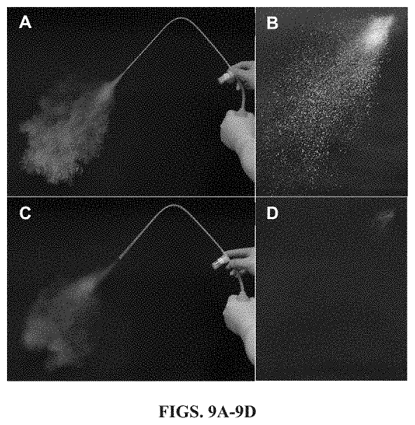

The powder prepared with lactose/leucine formed a denser plume (FIG. 9A) compared to that prepared with sucrose (FIG. 9C). Additionally, the powder prepared with lactose/leucine was substantially more visibly dispersed on the poster surface (FIG. 9B) than was the powder prepared with sucrose (FIG. 9D). Of note, almost all of the powder prepared with lactose/leucine was ejected from the vial, while about only ⅔ of the powder prepared with sucrose was ejected and the remaining ⅓ was trapped at the top of the vial. The ejection efficiency may be modified by conditioning the powder before insufflation.

C. Discussion

Both compositions were able to be aerosolized from the NOVATECH® TALCAIR™ powder blower, a device that is FDA-approved to deliver Talc powder to the intrapleural space. The lactose/leucine composition led to a more easily aerosolized powder that dispersed well onto the poster, while the sucrose formulation was less easily dispersed (i.e., ejected from the vial) and did not visibly adhere to the poster. Therefore, the composition of the formulation plays an important role in dictating the aerosol performance and deposition. Overall, this demonstrates the potential for the TFF powders to be administered to the intrapleural space or any macroscopically and microscopically exposed tissue surface and implies that some compositions can stick to nearby surfaces upon aerosolization, providing a homogenous distribution at the site.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Baughman et al., Am. J. Respir. Crit. Care Med. 2006, 174 (7), 795-802.
Bhambhani and Blue, Am. Pharm. Rev., 2010, 13 (1), 31-38.
Boulet et al., Am. J. Respir. Crit. Care Med. 1997, 155 (6), 1835-1840.
Breen et al., Pharm. Res. 2001, 18 (9), 1345-1353.
Cheng et al., Lung, 2000, 178 (1), 19-29.
Engstrom et al., Pharm. Res. 2008, 25 (6), 1334-1346.
Emig et al., BioRxiv, 2021, doi.org/10.1101/2021.10.12.464150
Faghihi et al., AAPS Pharm Sci Tech 2019, 20 (3), 1-9.
Fahy et al., Am. J. Respir. Crit. Care Med 1999, 160 (3), 1023-1027.
Guilleminault et al., J. Control. Release 2014, 196, 344-54.
Karampitsakos et al., Front. Med., 2019, 6, 41.
Li et al., The AAPS journal 2019, 21 (3), 1-17.
Liang et al., Pharmaceutics 2020, 12 (11), 1025.
Maillet et al., Pharm. Res. 2011, 28 (9), 2147-56.
Merck, Keytruda package insert, 2014.
Raghu et al., Am. J. Respir. Crit. Care Med. 2008, 178 (9), 948-955.
Rossman et al., Sarcoidosis. Vasc. Defuse Lung Dis. 2006, 23 (3), 201-208.
Sweiss et al., Sarcoidosis, vasculitis, and diffuse lung diseases: official journal of WASOG, World Association of Sarcoidosis and Other Granulomatous Disorders 2014, 31 (1), 46.

Utz et al., Chest 2003, 124 (1), 177-185.

Watts et al., Pharm. Res. 2013, 30 (3), 813-25.

1. A pharmaceutical composition comprising a plurality of drug particles, wherein each drug particle comprises:
(A) an antibody or an antibody fragment;
(B) a sugar or sugar alcohol; and
(C) an amino acid;
wherein the pharmaceutical composition is formulated as a dry powder.

2-3. (canceled)

4. The pharmaceutical composition according to claim 1, wherein the dry powder is formulated for administration to the lungs.

5. The pharmaceutical composition of claim 4, wherein the administration to the lungs is administration by oral inhalation.

6. The pharmaceutical composition according to claim 1, wherein the sugar is a cyclodextran.

7. The pharmaceutical composition of claim 6, wherein the surface is a nasal mucosal surface, an oral mucosal surface, the surface of tumor tissue, a vaginal mucosal surface, skin, an intrapleural space, or a surgical site.

8. The pharmaceutical composition according to claim 1, wherein the dry powder is reconstituted into a liquid.

9. (canceled)

10. The pharmaceutical composition of claim 8, wherein the liquid is formulated for use as an intravenous injection, as a subcutaneous injection, in nebulization to the lungs, or in spraying into the nasal cavity.

11. The pharmaceutical composition according to claim 1, further comprising a buffer.

12-14. (canceled)

15. The pharmaceutical composition of claim 1, wherein the sugar is lactose, trehalose, or sucrose.

16-18. (canceled)

19. The pharmaceutical composition according to claim 1, wherein the amino acid is a canonical amino acid or a non-polar amino acid.

20-23. (canceled)

24. The pharmaceutical composition according to claim 1, wherein the antibody is a monoclonal antibody: an IgG antibody: an anti-CTL4A antibody: an anti-PD-L1 antibody: or an anti-TNF-α antibody.

25-32. (canceled)

33. The pharmaceutical composition according to claim 1, wherein the pharmaceutical composition comprises a weight:weight ratio of the sugar to the amino acid from about 1:6 to about 9:1.

34-37. (canceled)

38. The pharmaceutical composition according to claim 1, wherein the pharmaceutical composition comprises a weight ratio from about 0.1% to about 80% of the antibody relative to the total amount of the pharmaceutical composition.

39-48. (canceled)

49. The pharmaceutical composition according to claim 1, wherein the drug particles have a mass median aerodynamic diameter (MMAD) from about 0.5 μm to about 25.0 μm.

50-53. (canceled)

54. The pharmaceutical composition according to claim 1, wherein the drug particles have a geometric standard deviation (GSD) from about 1.0 to about 5.0.

55-57. (canceled)

58. The pharmaceutical composition according to claim 1, wherein the pharmaceutical composition is formulated into a capsule for use in a dry powder inhaler.

59. The pharmaceutical composition according to claim 1, wherein the pharmaceutical composition is formulated into an inhaler.

60-78. (canceled)

79. The pharmaceutical composition according to claim 45, wherein the storage temperature is from about −0° C. to about 25° C.

80-88. (canceled)

89. A method of preparing a pharmaceutical composition according to claim 1 comprising:
(A) dissolving:
(1) an antibody or antibody fragment;
(2) a sugar or sugar alcohol; and
(3) mucoadhesive polymer;
in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

90-164. (canceled)

165. A pharmaceutical composition comprising a plurality of drug particles; wherein each drug particle comprises:
(A) an antibody or antibody fragment;
(B) trehalose; and
(C) leucine;
wherein the pharmaceutical composition is formulated for administration to the lungs, and has a mass median aerodynamic diameter (MMAD) from about 1.0 μm to about 4.0 μm.

166-172. (canceled)
